SEQUENCE LISTING (133 sequences)

SEQ ID NO: 1 - protein, 119 aa, Artificial Sequence
  heavy chain variable region
QVQLKESGPDLVAPSQSLSITCTVSGFSLTNYGVHWVRQPPGKGLEWLVVIWSDGRINYNSALKSRLSITKDNSKRQVFLKMNSLQIDDTAIYYCVRHPAFGPHAMDYWGQGISVTVSS

SEQ ID NO: 2 - protein, 112 aa, Artificial Sequence
  light chain variable region
DIVMTQDAPSIPVTPGESVSISCRSSKSLLNSNGNTYLYWFLQRPGQSPHLLLYRMSNPASGAPDRFSGSGSGTEFTLRISRVEAEDVGVYYCMQYLEYPPTFGAGTRLELK

SEQ ID NO: 3 - protein, 117 aa, Artificial Sequence
  heavy chain variable region
EVMLVESGGGLVRPGGSLKLSCAGSGITLSTYAMSWVRQTPERRLEWVASIISGGITYYLDSVKGRFTISRDNARNILYLQMSSLRSEDTAIYYCARKYHGNALDYWGQGTSVTVSL

SEQ ID NO: 4 - protein, 113 aa, Artificial Sequence
  light chain variable region
DIVMTQSPSSLPVTAGETVTMRCKSSQSLLNSGNQRNYLTWYQRKPGQPPKKLIYWASTRESGVPDRFTGSGSGTDFTLTISGVQAEDLAVYYCQNNYFYPLTFGAGTKLELK

SEQ ID NO: 5 - protein, 118 aa, Artificial Sequence
  heavy chain variable region
QIQMVQSGPELKKPGETVRISCKASGYSFTTAGMQWVRKMPGEGLKWIGWIIAHSGEPKYTEDFKGRFAFSLETSASTTYLQISNLKNEDTATYFCARWGKGNTMDYWGQGTSVIVSS

SEQ ID NO: 6 - protein, 113 aa, Artificial Sequence
  light chain variable region
DIVMTQSPSSLTVTAGEKVTMSCKSSQSLLNGGNQRNYLTWYQQKPGQPPKLLIYWASTRESGVPDRFTGSGSGTHFTLTISSVQAEDLAVYYCQNAYFFPLTFGAGTKLELK

SEQ ID NO: 7 - protein, 119 aa, Artificial Sequence
  heavy chain variable region
DVQLVESGGGLVQPGGSRKLSCAASGFTFSSFGMHWVRQAPEKGLEWVAYISSGSSTIYYPDTVKGRFTVSRDNPKNTLFLQMTSLRSEDTAMYYCVRLGPRGNVMDHWGQGTSVTVSS

SEQ ID NO: 8 - protein, 113 aa, Artificial Sequence
  light chain variable region
DIVMTQSPSSLTVTAGEKVTMSCKSSQSLLNSGNQRNYLTWYQQKPGQPPKLLIYWASTRESGVPDRFTGSGSGTDFTLTISSVQAEDLAVYYCQNVYFYPLTFGTGTKLELR

SEQ ID NO: 9 - protein, 118 aa, Artificial Sequence
  heavy chain variable region
DVQLQESGPDLVKPSQSLSLTCTVSGYSISGAYNWHWIRQFPGNKLEWLAYMQYSGSSNYNPSFKSRISISRDTSKNQFFLQLKSVTTEDTATYYCARMYNGNSFLYWGQGTLVTVSA

SEQ ID NO: 10 - protein, 113 aa, Artificial Sequence
  light chain variable region
DIVMTQSPSSLTVTAGEKVTMNCKSSQSLFNSGNQKNYLTWYQQKPGQPPRLLIYWASTRESGVPDRFTGSGSGTDFTLTISSVQAEDLSLYYCQNSYSYPLTFGAGTKLELK

SEQ ID NO: 11 - protein, 119 aa, Artificial Sequence
  heavy chain variable region
QVQLKESGPDLVAPSQSLSLTCSVSGFSLTSYGIHWVRQPPGKGLEWLVVIWSDGRTTYNSGLKSRLSISKDNSKSQVLLKMNSLRTDDTAIYYCVRHPAFGPHAMDYWGQGTSVTVSS

SEQ ID NO: 12 - protein, 112 aa, Artificial Sequence
  light chain variable region
DIVMTQAAPSVPVTPGESVSISCRSSKSLLNSNGNTYLYWFLQRPGQSPQLLIYRMSNLASGVPDRFSGSGSGTDFTLRISRVEAGDVGVYYCMQYLEYPVTFGAGTKLELK

SEQ ID NO: 13 - protein, 119 aa, Artificial Sequence
  heavy chain variable region
DVQLVESGGGLVQPGGSRKLSCAASGFTFSRFGMHWVRQAPKKGLEWVAYISSGSNTIYYADTVKGRFTISRDNPKNTLFLQTTSLRSEDTAIYYCGRLGFYGNSFDHWGQGTLVTVSA

SEQ ID NO: 14 - protein, 113 aa, Artificial Sequence
  light chain variable region
NILMTQSPSSLTVTAGEKVTMNCKSSQSLLNGGNQRNYLTWYQQKAGQPPKLLIYWASTRESGVPDRFTGGGSGTDFTLTISSVQAEDLALYYCQNSYYYPLTFGAGTKLELK

SEQ ID NO: 15 - protein, 118 aa, Artificial Sequence
  heavy chain variable region
EVQLRQSGPELVKPGASVKMSCKASGYTFTTYIINWVKQKPGQGLEWIGYINPYNDDTRYNERVKGKATLTSDKSSSTAYMELSSLTSEDSAVYYCARFYFGNSFTYWGQGTLVTVSA

SEQ ID NO: 16 - protein, 113 aa, Artificial Sequence
  light chain variable region
DIVMTQSPSSLPVTVGERVTMTCKSSQGLFNSGNQRNYLTWYQQKPGQPPKLLIYWASTRESGVPDRFTGSGSGTDFTLTISSVQAEDLAIYYCQNNYIYPLTFGAGTKLELK

SEQ ID NO: 17 - protein, 118 aa, Artificial Sequence
  heavy chain variable region
EVQLRQSGPELVKPGASVKMSCKASGYTFTTYIINWVKQKPGQGLEWIGYINPYNDGTRYNERVKGKATLTSDKSSSTAYMELSSLTSEDSAVYYCARFHFGNSFTYWGQGTLVTVSA

SEQ ID NO: 18 - protein, 113 aa, Artificial Sequence
  light chain variable region
DIVMTQSPSSLPVTTGEKVTMTCKSSQGLFNNGNQRNYLTWYQQKPGQPPKLLIYWASTRESGVPDRFIGSGSGTDFTLTISSVQAEDLAIYYCQNNYIFPLTFGAGTKLELK

SEQ ID NO: 19 - protein, 118 aa, Artificial Sequence
  heavy chain variable region
QIQLVQSGPELKKPGETVKISCKASGYTLTNYGMNWVRQAPGKGLKWMGWIRPNTGEPTYAEDFKGRFVFSLETSAATAYLQITNLKSEDTSTYFCARLYRGNTLDNWGQGTSVIVSS

SEQ ID NO: 20 - protein, 113 aa, Artificial Sequence
  light chain variable region
DIVMTQSPSSLTVTTGEKVTMSCKSSQNLLNSGNQRNYLTWYQQKPGQSPKLLIYWASTRESGVPYRFTGSGSGTDFTLTISSVQTDDLAIYYCQNGYSFPFTFGSGTKLEIK

SEQ ID NO: 21 - protein, 118 aa, Artificial Sequence
  heavy chain variable region
QVHLQQSGAELVRPGSSVKISCKASGYAFSNYWMNWVRQRPGQGLEWIGQIYPGNGDTKYSGKFNSKDTLTADKSSNTAYMQLNSLTSEDSAVYFCARFYYGNVMDYWGQGTSVTVSS

SEQ ID NO: 22 - protein, 113 aa, Artificial Sequence
  light chain variable region
DIVLTQSPSSLTVTAGEKVTMSCKSSQTLLNGGNQKNYLTWYQQKSGQPPKLLIYWASTRESGVPDRFTGSGSGTDFTLTISSVQAEDLAVYYCQNGYSYPLTFGVGTKLELK

SEQ ID NO: 23 - protein, 117 aa, Artificial Sequence
  heavy chain variable region
EVQLVESGGGLVQPGGSLRLSCAGSGITLSTYAMSWVRQAPGKGLEWVSSIISGGITYYLDSVKGRFTISRDNAKNTLYLQMSSLRAEDTAVYYCARKYHGNALDYWGQGTLVTVSS

SEQ ID NO: 24 - protein, 113 aa, Artificial Sequence
  light chain variable region
DIVMTQSPDSLAVSLGERATINCKSSQSLLSSGNQRNYLTWYQQKPGQPPKKLIYWASTRESGVPDRFSGSGSGTDFTLTISSLQAEDLAVYYCQNNYFYPLTFGQGTKLEIK

SEQ ID NO: 25 - protein, 113 aa, Artificial Sequence
  light chain variable region
DIVMTQSPDSLAVSLGERATINCKSSQSLLSSGNQRNYLTWYQQKPGQPPKKLIYWASTRESGVPDRFSGSGSGTDFTLTISSLQAEDLAVYYCQSNYFYPLTFGQGTKLEIK

SEQ ID NO: 26 - protein, 113 aa, Artificial Sequence
  light chain variable region
DIVMTQSPDSLAVSLGERATINCKSSQSLLSSGNQRNYLTWYQQKPGQPPKKLIYWASTRESGVPDRFSGSGSGTDFTLTISSLQAEDLAVYYCQQNYFYPLTFGQGTKLEIK

SEQ ID NO: 27 - protein, 118 aa, Artificial Sequence
  heavy chain variable region
QIQLVQSGSELKKPGASVKVSCKASGYTLTNYGMNWVRQAPGQGLEWMGWIRPNTGEPTYAEDFKGRFVFSLDTSVATAYLQITSLKAEDTAVYYCARLYRGNTLDNWGQGTLVTVSS

SEQ ID NO: 28 - protein, 113 aa, Artificial Sequence
  light chain variable region
DIVMTQSPDSLAVSLGERATINCKSSQNLLSSGNQRNYLTWYQQKPGQPPKLLIYWASTRESGVPDRFSGSGSGTDFTLTISSLQAEDLAVYYCQQGYSFPFTFGQGTKLEIK

SEQ ID NO: 29 - protein, 118 aa, Artificial Sequence
  heavy chain variable region
QVQLVQSGAEVKKPGSSVKVSCKASGYAFSNYWMNWVRQAPGQGLEWMGQIYPGSGDTKYSGKFQSRVTITADKSTNTAYMELSSLRSEDTAVYYCARFYYGNVMDYWGQGTLVTVSS

SEQ ID NO: 30 - protein, 113 aa, Artificial Sequence
  light chain variable region
DIVLTQSPDSLAVSLGERATINCKSSQTLLSGGNQKNYLTWYQQKPGQPPKLLIYWASTRESGVPDRFSGSGSGTDFTLTISSLQAEDLAVYYCQQGYSYPLTFGQGTKLEIK

SEQ ID NO: 31 - protein, 10 aa, Artificial Sequence
  heavy chain, CDR1
GFSLTNYGVH

SEQ ID NO: 32 - protein, 17 aa, Artificial
  heavy chain, CDR2
VVIWSDGRINYNSALKS

SEQ ID NO: 33 - protein, 9 aa, Artificial Sequence
  heavy chain, CDR3
AFGPHAMDY

SEQ ID NO: 34 - protein, 16 aa, Artificial Sequence
  light chain, CDR1
RSSKSLLNSNGNTYLY

SEQ ID NO: 35 - protein, 7 aa, Artificial Sequence
  light chain, CDR2
RMSNPAS

SEQ ID NO: 36 - protein, 9 aa, Artificial Sequence
  light chain, CDR3
MQYLEYPPT

SEQ ID NO: 37 - protein, 10 aa, Artificial Sequence
  heavy chain, CDR1
GITLSTYAMS

SEQ ID NO: 38 - protein, 16 aa, Artificial Sequence
  heavy chain, CDR2
SIISGGITYYLDSVKG

SEQ ID NO: 39 - protein, 9 aa, Artificial Sequence
  heavy chain, CDR3
KYHGNALDY

SEQ ID NO: 40 - protein, 17 aa, Artificial Sequence
  light chain, CDR1
KSSQSLLNSGNQRNYLT

SEQ ID NO: 41 - protein, 7 aa, Artificial Sequence
  light chain, CDR2
WASTRES

SEQ ID NO: 42 - protein, 9 aa, Artificial Sequence
  light chain, CDR3
QNNYFYPLT

SEQ ID NO: 43 - protein, 10 aa, Artificial Sequence
  heavy chain, CDR1
GYSFTTAGMQ

SEQ ID NO: 44 - protein, 18 aa, Artificial Sequence
  heavy chain, CDR2
GWIIAHSGEPKYTEDFKG

SEQ ID NO: 45 - protein, 9 aa, Artificial Sequence
  heavy chain, CDR3
WGKGNTMDY

SEQ ID NO: 46 - protein, 17 aa, Artificial Sequence
  light chain, CDR1
KSSQSLLNGGNQRNYLT

SEQ ID NO: 47 - protein, 9 aa, Artificial Sequence
  light chain, CDR3
QNAYFFPLT

SEQ ID NO: 48 - protein, 10 aa, Artificial Sequence
  heavy chain, CDR1
GFTFSSFGMH

SEQ ID NO: 49 - protein, 18 aa, Artificial Sequence
  heavy chain, CDR2
AYISSGSSTIYYPDTVKG

SEQ ID NO: 50 - protein, 8 aa, Artificial Sequence
  heavy chain, CDR3
PRGNVMDH

SEQ ID NO: 51 - protein, 9 aa, Artificial Sequence
  light chain, CDR3
QNVYFYPLT

SEQ ID NO: 52 - protein, 11 aa, Artificial Sequence
  heavy chain, CDR1
GYSISGAYNWH

SEQ ID NO: 53 - protein, 17 aa, Artificial Sequence
  heavy chain, CDR2
AYMQYSGSSNYNPSFKS

SEQ ID NO: 54 - protein, 9 aa, Artificial Sequence
  heavy chain, CDR3
MYNGNSFLY

SEQ ID NO: 55 - protein, 17 aa, Artificial Sequence
  light chain, CDR1
KSSQSLFNSGNQKNYLT

SEQ ID NO: 56 - protein, 9 aa, Artificial Sequence
  light chain, CDR3
QNSYSYPLT

SEQ ID NO: 57 - protein, 10 aa, Artificial Sequence
  heavy chain, CDR1
GFSLTSYGIH

SEQ ID NO: 58 - protein, 17 aa, Artificial Sequence
  heavy chain, CDR2
VVIWSDGRTTYNSGLKS

SEQ ID NO: 59 - protein, 7 aa, Artificial Sequence
  light chain, CDR2
RMSNLAS

SEQ ID NO: 60 - protein, 9 aa, Artificial Sequence
  light chain, CDR3
MQYLEYPVT

SEQ ID NO: 61 - protein, 10 aa, Artificial Sequence
  heavy chain, CDR1
GFTFSRFGMH

SEQ ID NO: 62 - protein, 18 aa, Artificial Sequence
  heavy chain, CDR2
AYISSGSNTIYYADTVKG

SEQ ID NO: 63 - protein, 8 aa, Artificial Sequence
  heavy chain, CDR3
FYGNSFDH

SEQ ID NO: 64 - protein, 9 aa, Artificial Sequence
  light chain, CDR3
QNSYYYPLT

SEQ ID NO: 65 - protein, 10 aa, Artificial Sequence
  heavy chain, CDR1
GYTFTTYIIN

SEQ ID NO: 66 - protein, 18 aa, Artificial Sequence
  heavy chain, CDR2
GYINPYNDDTRYNERVKG

SEQ ID NO: 67 - protein, 9 aa, Artificial Sequence
  heavy chain, CDR3
FYFGNSFTY

SEQ ID NO: 68 - protein, 17 aa, Artificial Sequence
  light chain, CDR1
KSSQGLFNSGNQRNYLT

SEQ ID NO: 69 - protein, 9 aa, Artificial Sequence
  light chain, CDR3
QNNYIYPLT

SEQ ID NO: 70 - protein, 18 aa, Artificial Sequence
  heavy chain, CDR2
GYINPYNDGTRYNERVKG

SEQ ID NO: 71 - protein, 9 aa, Artificial Sequence
  heavy chain, CDR3
FHFGNSFTY

SEQ ID NO: 72 - protein, 17 aa, Artificial Sequence
  light chain, CDR1
KSSQGLFNNGNQRNYLT

SEQ ID NO: 73 - protein, 9 aa, Artificial Sequence
  light chain, CDR3
QNNYIFPLT

SEQ ID NO: 74 - protein, 10 aa, Artificial Sequence
  heavy chain, CDR1
GYTLTNYGMN

SEQ ID NO: 75 - protein, 17 aa, Artificial Sequence
  heavy chain, CDR2
WIRPNTGEPTYAEDFKG

SEQ ID NO: 76 - protein, 9 aa, Artificial Sequence
  heavy chain, CDR3
LYRGNTLDN

SEQ ID NO: 77 - protein, 17 aa, Artificial Sequence
  light chain, CDR1
KSSQNLLNSGNQRNYLT

SEQ ID NO: 78 - protein, 9 aa, Artificial Sequence
  light chain, CDR3
QNGYSFPFT

SEQ ID NO: 79 - protein, 10 aa, Artificial Sequence
  heavy chain, CDR1
GYAFSNYWMN

SEQ ID NO: 80 - protein, 18 aa, Artificial Sequence
  heavy chain, CDR2
GQIYPGNGDTKYSGKFNS

SEQ ID NO: 81 - protein, 9 aa, Artificial Sequence
  heavy chain, CDR3
FYYGNVMDY

SEQ ID NO: 82 - protein, 17 aa, Artificial Sequence
  light chain, CDR1
KSSQTLLNGGNQKNYLT

SEQ ID NO: 83 - protein, 9 aa, Artificial Sequence
  light chain, CDR3
QNGYSYPLT

SEQ ID NO: 84 - protein, 119 aa, Artificial Sequence
  heavy chain variable region
QVQLVESGGGVVQPGRSLRLSCAASGFSLTNYGVHWVRQAPGKGLEWVAVIWSDGRINYNSALKSRFTISRDNSKNTLYLQMNSLRAEDTAVYYCARHPAFGPHAMDYWGQGTLVTVSS

SEQ ID NO: 85 - protein, 17 aa, Artificial Sequence
  light chain, CDR1
KSSQSLLSSGNQRNYLT

SEQ ID NO: 86 - protein, 9 aa, Artificial Sequence
  light chain, CDR3
QSNYFYPLT

SEQ ID NO: 87 - protein, 9 aa, Artificial Sequence
  light chain, CDR3
QQNYFYPLT

SEQ ID NO: 88 - protein, 112 aa, Artificial Sequence
  light chain variable region
DIVMTQSPLSLPVTPGEPASISCRSSKSLLNSNGNTYLYWYLQKPGQSPQLLIYRMSNPASGVPDRFSGSGSGTDFTLKISRVEAEDVGVYYCMQYLEYPPTFGAGTKLEIK

SEQ ID NO: 89 - protein, 17 aa, Artificial Sequence
  light chain, CDR1
KSSQNLLSSGNQRNYLT

SEQ ID NO: 90 - protein, 9 aa, Artificial Sequence
  light chain, CDR3
QQGYSFPFT

SEQ ID NO: 91 - protein, 17 aa, Artificial Sequence
  heavy chain, CDR2
QIYPGSGDTKYSGKFQS

SEQ ID NO: 92 - protein, 17 aa, Artificial Sequence
  light chain, CDR1
KSSQTLLSGGNQKNYLT

SEQ ID NO: 93 - protein, 9 aa, Artificial Sequence
  light chain, CDR3
QQGYSYPLT

SEQ ID NO: 94 - DNA, 357 bp, Artificial Sequence
  heavy chain variable region
caggtgcagctgaaggagtcaggacctgacctggtggcgccctcacagagcctgtccatcacatgcaccgtctcagggttctccttaaccaactatggtgtacactgggttcgccagcctccaggaaagggtctggaatggctggtagtgatatggagtgatggaagaataaactataattcagctctcaaatccagactgagcatcaccaaggacaattccaagaggcaagttttcctaaaaatgaacagtctccaaattgatgacacagccatttattattgtgtcagacatcccgccttcggtccccatgctatggactactggggtcaaggaatctcagtcaccgtctcctca

SEQ ID NO: 95 - DNA, 336 bp, Artificial Sequence
  light chain variable region
gatattgtgatgactcaggatgcaccctctatacctgtcactcctggagagtcagtatccatctcctgcaggtctagtaagagtctcctgaatagtaatggcaacacttacttatattggttcttgcagaggccaggccagtctcctcacctcctgctttatcggatgtccaaccctgcctcaggagccccagacaggttcagtggcagtgggtcagggactgaattcacactgagaatcagtagagtggaggctgaagatgtgggtgtttattactgtatgcaatatctggaatatccgcccacgttcggtgctgggaccaggctggagctgaaa

SEQ ID NO: 96 - DNA, 352 bp, Artificial Sequence
  heavy chain variable region
gaagtgatgctggtggagtctgggggaggcttagtgaggcctggagggtccctgaaactctcctgtgcaggctctggaatcactctcagtacctatgccatgtcttgggttcgccagactccagagaggaggctggagtgggtcgcatccattattagtggtggtatcacctattatttagacagtgtgaagggccgcttcaccatctccagagataatgccaggaacatcctgtacctgcaaatgagcagtctgaggtctgaggacacggccatttattactgtgcaagaaaataccacgggaatgctttggactactggggtcaaggaacctcagtcaccgtctctttag

SEQ ID NO: 97 - DNA, 340 bp, Artificial Sequence
  light chain variable region
gacattgtgatgacacagtctccatcgtccctgcctgtgacagcaggagagacggtcactatgcgctgcaagtccagtcagagtcttttaaacagtggaaatcaaaggaactacttgacctggtaccagcggaaaccaggacagcctcctaagaaactgatctactgggcatccactagggaatctggggtccctgatcgcttcacaggcagtggatctggaacagatttcactctcaccatcagtggtgtgcaggctgaagacctggcagtttattactgtcagaataattatttttatccgctcacgttcggtgctgggaccaagctggagctgaaac

SEQ ID NO: 98 - DNA, 354 bp, Artificial Sequence
  heavy chain variable region
cagatccagatggtgcagtctggacctgagctgaagaagcctggagagacagtcaggatctcctgcaaggcttctgggtattccttcacaactgctggaatgcagtgggtgcgaaagatgcctggagagggtttgaagtggattggctggataatcgcccactctggagagccaaaatatacagaagacttcaagggacggtttgccttctctttggaaacctctgccagcactacatatttacagataagtaacctcaaaaatgaggacacggctacgtatttctgtgcgagatggggaaagggaaatactatggactattggggtcaaggaacctcagtcatcgtctcctca

SEQ ID NO: 99 - DNA, 339 bp, Artificial Sequence
  light chain variable region
gacattgtgatgacacagtcgccatcctccctgactgtgacagcaggagagaaggtcactatgagctgcaagtccagtcagagtctgttaaacggtggaaatcagaggaactacttgacctggtaccagcagaaaccaggacagcctcctaaacttttgatctactgggcatccactagggaatctggggtccctgatcgcttcacaggcagtggatctggaacacatttcactctcaccatcagcagtgtgcaggctgaagacctggcagtttattactgtcagaatgcttatttttttccgctcacgttcggtgctgggaccaagctagagctgaaa

SEQ ID NO: 100 - DNA, 358 bp, Artificial Sequence
  heavy chain variable region
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccatctactatccagacacagtgaagggccgattcaccgtctccagagacaatcccaagaacaccctgttcctgcaaatgaccagtctaaggtctgaggacacggccatgtattactgtgtaagattgggtccacgggggaatgttatggaccattggggtcaaggaacctcagtcaccgtctcctcag

SEQ ID NO: 101 - DNA, 339 bp, Artificial Sequence
  light chain variable region
gacattgtgatgacacagtctccatcctccctgactgtgacagcaggagagaaggtcactatgagctgcaagtccagtcagagtctgttaaacagtggaaatcaaaggaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgtttatttttatccactcacgttcggtactgggaccaagctggagctgaga

SEQ ID NO: 102 - DNA, 354 bp, Artificial Sequence
  heavy chain variable region
gatgtgcagcttcaggagtcaggacctgacctggtaaaaccctctcagtcactttcactcacctgcactgtcagtggctactccatcagcggtgcttataactggcactggatccggcagtttccaggaaacaaactggagtggctggcctacatgcagtacagtggtagctctaactacaacccatctttcaaaagtcgaatctctatcagtcgagacacgtccaagaaccaattcttcctgcagttgaaatctgtgactactgaagacacagccacatattactgtgcaagaatgtataacggaaactcgtttctttactggggccaagggactctggtcactgtctctgca

SEQ ID NO: 103 - DNA, 339 bp, Artificial Sequence
  light chain variable region
gacattgtgatgacacagtctccatcctccctgactgtgacagcaggcgagaaggtcactatgaactgcaagtccagtcagagtctgtttaacagtggaaatcaaaagaactatttgacctggtatcagcaaaaaccagggcagcctcctagattgttgatctattgggcatccactagggaatctggggtccctgatcgcttcacaggcagtggatctggaacagatttcactctcaccatcagcagtgtgcaggctgaagacctgtcactttattactgtcagaattcttatagttatcctctcacgttcggtgctgggaccaagctggagttgaaa

SEQ ID NO: 104 - DNA, 357 bp, Artificial Sequence
  heavy chain variable region
caggtgcagctgaaggagtcaggacctgacctggtggcgccctcacagagcctgtccctcacatgctccgtctcagggttctccttaaccagctatggtattcactgggttcgccagcctccaggaaagggtctggagtggctggtggtgatatggagtgatggaagaacaacctataattcaggtctcaaatccagactgagcatcagcaaggacaactccaagagccaagttctcttaaaaatgaacagtctccgaactgatgacacagccatttactactgtgtcagacatcccgccttcggtccccatgctatggactactggggtcaaggaacctcagtcaccgtctcctca

SEQ ID NO: 105 - DNA, 336 bp, Artificial Sequence
  light chain variable region
gatattgtgatgactcaggctgcaccctctgtacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgatttcacactgagaatcagtagagtggaggctggggatgtgggtgtttattactgtatgcaatatctagaatatccggtcacgttcggtgctgggaccaagctggagctgaaa

SEQ ID NO: 106 - DNA, 357 bp, Artificial Sequence
  heavy chain variable region
gatgtgcagctggtggagtctgggggaggcttagtgcagcctggagggtcccggaaactctcctgtgcagcctctggattcactttcagtcgctttggaatgcactgggttcgtcaggctccaaaaaagggactggagtgggtcgcatacattagtagtggcagtaataccatctactatgcagacacagtgaagggccgattcaccatctccagagacaatcccaagaacaccctgttcctgcaaacgaccagtctaaggtctgaggacacggccatatattactgtggaagattgggcttctatggtaactcgtttgatcactggggccaagggactctggtcactgtctctgca

SEQ ID NO: 107 - DNA, 339 bp, Artificial Sequence
  light chain variable region
aacattttgatgacacagtctccatcctccctgactgtgacagcgggagagaaggtcactatgaactgcaagtccagtcagagtctgttaaacggtggaaatcaaaggaactacttgacctggtaccagcagaaagcagggcagcctcccaaactgttgatctactgggcatccactagggagtctggggtccctgatcgcttcacaggcggtggatctggaacagatttcactctcaccatcagcagtgtgcaggctgaagacctggcactttattactgtcagaattcttattattatccgctcacgttcggtgctggtaccaagctggagctgaaa

SEQ ID NO: 108 - DNA, 354 bp, Artificial Sequence
  heavy chain variable region
gaggtccagctgcggcagtctggacctgagctggtaaagcctggggcttcagtgaagatgtcctgcaaggcttctggatacacattcactacctatattataaactgggtgaagcagaagcctgggcagggccttgagtggattggatatattaatccttacaatgatgatacaaggtacaatgagagggtcaaaggcaaggccacactgacttcagacaaatcctccagcactgcctacatggagctcagcagcctgacctctgaagactctgcggtctattactgtgcaagattttactttggtaactcgtttacttactggggccaagggactctggtcactgtctctgca

SEQ ID NO: 109 - DNA, 339 bp, Artificial Sequence
  light chain variable region
gacattgtgatgacacagtctccgtcctccctgcctgtgacagtaggagagagggtcactatgacctgcaagtccagtcagggtctgtttaacagtggaaatcaaaggaactacttgacctggtaccaacagaaaccagggcagcctcctaaactgttgatctactgggcatccactagggaatctggggtccctgatcgcttcacaggcagtggatctggaacagatttcactctcaccatcagcagtgtgcaggctgaagacctggcaatttattactgtcagaataattatatttatccgctcacgttcggtgctgggaccaagttggagctgaaa

SEQ ID NO: 110 - DNA, 354 bp, Artificial Sequence
  heavy chain variable region
gaggtccagctgcggcagtctggacctgagctggtaaagcctggggcttcagtgaagatgtcctgcaaggcttctggatacacattcactacctatattataaactgggtgaagcagaagcctgggcagggccttgagtggattggatatattaatccttacaatgatggtactaggtacaatgagagggtcaaaggcaaggccacactgacttcagacaaatcctccagtacagcctacatggagctcagcagcctgacctctgaggactctgcggtctactactgtgcaagatttcactttggtaattcgtttacttactggggccaagggactctggtcactgtctctgca

SEQ ID NO: 111 - DNA, 339 bp, Artificial Sequence
  light chain variable region
gacattgtgatgacacagtctccgtcctccctgcctgtgacaacaggagagaaggtcactatgacttgcaagtccagtcagggtctgtttaacaatggaaatcaaaggaactacttgacctggtaccagcagaaaccagggcagcctcctaaactgttgatctactgggcatccactagggaatctggggtccctgatcgcttcataggcagtggatctggaacagatttcactctcaccatcagcagtgtgcaggctgaagacctggcaatttattactgtcagaataattatatttttccgctcacgttcggtgctgggaccaagttggagctgaaa

SEQ ID NO: 112 - DNA, 354 bp, Artificial Sequence
  heavy chain variable region
cagatccagttggtgcagtctggacctgagctgaagaagcctggagagacagtcaagatctcctgcaaggcttctggatataccctcacaaactatggaatgaattgggtgaggcaggctccaggaaagggtttaaagtggatgggctggataaggcccaacactggagagccaacatatgctgaagacttcaagggacgatttgtcttctctttggaaacctctgccgccactgcctatttacagatcaccaacctcaaaagtgaggacacgtctacatatttctgtgcaagactgtacaggggaaatactttggacaactggggtcaaggaacctcagtcatcgtctcctca

SEQ ID NO: 113 - DNA, 339 bp, Artificial Sequence
  light chain variable region
gacattgtgatgacacagtctccatcctccctgactgtgacaacaggggagaaggtcactatgagctgcaagtccagtcagaatctgttaaacagtggaaatcaaaggaactacttgacctggtaccagcagaagccaggacagtctcctaaactattgatctactgggcatccactagggaatctggggtcccttatcgcttcacaggcagtggatctggaacagatttcactctcaccatcagcagtgtacagactgatgacctggctatttattactgtcagaatggttatagttttccattcacgttcggctcggggacaaagttggaaataaaa

SEQ ID NO: 114 - DNA, 354 bp, Artificial Sequence
  heavy chain variable region
caggttcacctgcagcagtctggggctgaactggtgaggcctgggtcctcagtgaagatttcctgcaaggcttctggctatgcattcagtaattactggatgaactgggtgaggcagaggcctggacagggtcttgagtggattggacagatttatcctggaaatggtgatactaagtacagtggaaagttcaacagtaaagacacactgactgcagacaaatcctccaacacagcctacatgcaactcaacagcctaacatctgaggactctgcggtctatttctgtgcaagattttactacggtaatgttatggactactggggtcaaggaacctcagtcaccgtctcctcg

SEQ ID NO: 115 - DNA, 339 bp, Artificial Sequence
  light chain variable region
gacattgtgttgacacagtctccatcctccctgactgtgacagcaggagagaaggtcactatgagctgcaagtccagtcagactctgttaaacggtggaaatcaaaagaactacttgacctggtaccagcagaaatcagggcagcctcctaaactgttgatctattgggcatcaactagggaatctggggtccctgatcgcttcacaggcagtggatctggaacagatttcactctcaccattagcagtgtccaggctgaagacctggcagtttattactgtcagaatggttatagttatccgctcacgttcggtgttgggaccaagctggagctgaaa

SEQ ID NO: 116 - DNA, 351 bp, Artificial Sequence
  heavy chain variable region
gaggtgcagctggtggagagcggggggggactggtgcagccaggaggaagcctgagactgagctgtgccggcagtggcattaccctgagcacatatgccatgagctgggtgagacaggcccctggcaaaggactggagtgggtgagcagcattatcagcggaggaatcacctactatctggatagcgtgaagggacggttcacaatcagcagggataacgccaagaacacactgtatctgcagatgagctctctgagggccgaagacaccgccgtgtattattgcgccagaaagtaccacggaaacgccctggactactggggccaggggaccctggtgacagtgagctcc

SEQ ID NO: 117 - DNA, 339 bp, Artificial Sequence
  light chain variable region
gacattgtgatgacccagagccccgactccctggccgtgagcctgggagaaagagccaccatcaactgcaagagcagccagagcctgctgagcagcggcaaccagagaaactacctgacctggtaccagcagaaacccggccagccccccaaaaagctgatctactgggcctccacccgggaatccggcgtgccagacagattctccggctccggctccggaaccgacttcaccctgaccatcagctccctgcaggctgaggacctggccgtgtactactgccagaacaactacttctaccccctgaccttcggccagggcaccaaactggagatcaag

SEQ ID NO: 118 - DNA, 339 bp, Artificial Sequence
  light chain variable region
gacattgtgatgacccagagccccgactccctggccgtgagcctgggagaaagagccaccatcaactgcaagagcagccagagcctgctgagcagcggcaaccagagaaactacctgacctggtaccagcagaaacccggccagccccccaaaaagctgatctactgggcctccacccgggaatccggcgtgccagacagattctccggctccggctccggaaccgacttcaccctgaccatcagctccctgcaggctgaggacctggccgtgtactactgccagagcaactacttctaccccctgaccttcggccagggcaccaaactggagatcaag

SEQ ID NO: 119 - DNA, 339 bp, Artificial Sequence
  light chain variable region
gacattgtgatgacccagagccccgactccctggccgtgagcctgggagaaagagccaccatcaactgcaagagcagccagagcctgctgagcagcggcaaccagagaaactacctgacctggtaccagcagaaacccggccagccccccaaaaagctgatctactgggcctccacccgggaatccggcgtgccagacagattctccggctccggctccggaaccgacttcaccctgaccatcagctccctgcaggctgaggacctggccgtgtactactgccagcagaactacttctaccccctgaccttcggccagggcaccaaactggagatcaag

SEQ ID NO: 120 - DNA, 354 bp, Artificial Sequence
  heavy chain variable region
cagatccagctggtgcagagcggcagcgaactgaagaagcccggcgccagcgtgaaagtgagctgcaaggccagcggctacaccctgaccaactacggcatgaactgggtgagacaggctcccggccaggggctggaatggatgggatggatcagacccaacaccggagagccaacctacgccgaggacttcaagggcagattcgtgttcagcctggacacctctgtggccaccgcttacctgcagatcaccagcctgaaagccgaggacaccgccgtgtactactgtgcccgcctgtatagaggaaacaccctggacaactggggccagggcactctggtgaccgtgagctcc

SEQ ID NO: 121 - DNA, 339 bp, Artificial Sequence
  light chain variable region
gacattgtgatgacccagagccccgactccctggccgtgagcctgggagaaagagccaccatcaactgcaagagcagccagaacctgctgagcagcggcaaccagagaaactacctgacctggtaccagcagaaacccggccagccccccaaactgctgatctactgggcctccaccagagaaagcggcgtgcccgacagattctccggaagcggcagcggcaccgacttcaccctgactatcagcagcctgcaggctgaggacctggccgtgtactactgccagcagggctacagcttccccttcaccttcggccagggcaccaaactggagatcaaa

SEQ ID NO: 122 - DNA, 354 bp, Artificial Sequence
  heavy chain variable region
caggtgcagctggtgcagagcggcgccgaggtgaaaaagcccggctcctcagtgaaagtgagctgcaaggccagcggctacgccttctccaactactggatgaactgggtgagacaggccccaggccagggcctggaatggatgggacagatctaccccggcagcggcgatacaaagtacagcggaaagttccagagcagagtgaccatcaccgccgacaaaagcaccaacaccgcctacatggagctgagcagcctgagaagtgaggacaccgccgtgtactactgcgccagattctactacggcaacgtgatggactattggggccagggaaccctggtgaccgtgagctct

SEQ ID NO: 123 - DNA, 339 bp, Artificial Sequence
  light chain variable region
gacattgtgctgacccagagccccgactccctggctgtgagcctgggagagagagccaccatcaactgcaagagcagccagaccctgctgagtggaggaaaccagaaaaactacctgacttggtaccagcagaaacccggccagccccccaaactgctgatctactgggcctccacaagagaaagcggagtgcccgacagattcagcggcagcggcagcggaaccgacttcaccctgactatcagcagcctgcaggctgaggacctggccgtgtactactgccagcagggctacagctacccactgaccttcggacagggcaccaagctggagattaaa

SEQ ID NO: 124 - protein, 330 aa, Artificial Sequence
  heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 107 aa, Artificial Sequence [sequence omitted]

SEQ ID NO: 126 - protein, 448 aa, Artificial Sequence
  IMAB362 heavy chain
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 220 aa, Artificial Sequence
  IMAB362 light chain
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 113 aa, Artificial Sequence
  light chain variable region
DIVMTQSPDSLAVSLGERATINCKSSQSLLNSGNQRNYLTWYQQKPGQPPKLLIYWASTRESGVPDRFSGSGSGTDFTLTISSLQAEDVAVYYCQNNYFYPLTFGQGTKLEIK

SEQ ID NO: 129 - protein, 117 aa, Artificial Sequence
  heavy chain variable region
EVQLVESGGGLVQPGGSLRLSCAASGITLSTYAMSWVRQAPGKGLEWVSSIISGGITYYLDSVKGRFTISRDNAKNSLYLQMNSLRAEDTAVYYCARKYHGNALDYWGQGTLVTVSS

SEQ ID NO: 130 - protein, 113 aa, Artificial Sequence
  light chain variable region
DIVMTQSPDSLAVSLGERATINCKSSQNLLNSGNQRNYLTWYQQKPGQPPKLLIYWASTRESGVPDRFSGSGSGTDFTLTISSLQAEDVAVYYCQNGYSFPFTFGQGTKLEIK

SEQ ID NO: 131 - protein, 118 aa, Artificial Sequence
  heavy chain variable region
QVQLVQSGSELKKPGASVKVSCKASGYTLTNYGMNWVRQAPGQGLEWMGWIRPNTGEPTYAEDFKGRFVFSLDTSVSTAYLQICSLKAEDTAVYYCARLYRGNTLDNWGQGTLVTVSS

SEQ ID NO: 132 - protein, 113 aa, Artificial Sequence
  light chain variable region
DIVMTQSPDSLAVSLGERATINCKSSQTLLNGGNQKNYLTWYQQKPGQPPKLLIYWASTRESGVPDRFSGSGSGTDFTLTISSLQAEDVAVYYCQNGYSYPLTFGQGTKLEIK

SEQ ID NO: 133 - protein, 118 aa, Artificial Sequence
  heavy chain variable region
QVQLVQSGAEVKKPGSSVKVSCKASGYAFSNYWMNWVRQAPGQGLEWMGQIYPGNGDTKYSGKFNSRVTITADESTSTAYMELSSLRSEDTAVYYCARFYYGNVMDYWGQGTLVTVSS

(19) United States
(12) Patent Application Publication
(10) Pub. No.: US 2023/0250168 A1
(43) Pub. Date: Aug. 10, 2023

(54) ANTI-HUMAN CLAUDIN 18.2 ANTIBODY AND APPLICATION THEREOF

(71) Applicant: MABWELL (SHANGHAI) BIOSCIENCE CO., LTD., Shanghai (CN)

(72) Inventors: Jian LIN, Shanghai (CN); Xiaofang DENG, Shanghai (CN); Pan GAO, Shanghai (CN); Xiaohong XU, Shanghai (CN); Lichun WANG, Shanghai (CN); Hongyuan REN, Shanghai (CN); Jianjun BI, Shanghai (CN); Jin WANG, Shanghai (CN)

(21) Appl. No.: 17/755,427

(22) PCT Filed: Sep. 28, 2020
(86) PCT No.: PCT/CN2020/118424

§ 371 Date: Apr. 28, 2022

(30) Foreign Application Priority Data
Sep. 29, 2019 (CN) 201910929614.0

(51) Int. Cl. C07K 16/28; C12N 15/63; G01N 33/574; A61P 35/00

(52) U.S. Cl. CPC C07K 16/28; C12N 15/63; G01N 33/574; A61P 35/00; C07K 2317/565; C07K 2317/92; C07K 2317/24

(57) ABSTRACT

Embodiments of the present disclosure provide an antibody that binds to human Claudin 18.2 or a fragment thereof, as well as encoded nucleic acids and the like thereof. The anti-human Claudin 18.2 antibody of embodiments of the present disclosure has strong affinity to an antigen Claudin 18.2 and significant complement-dependent cytotoxicity (CDC) activity and antibody-dependent cytotoxicity (ADCC) activity to target expression cells, and exhibits high specificity to human CLDN 18.2.

### CROSS-REFERENCE TO RELATED APPLICATION(S)

This application is a U.S. National Phase Patent Application of International Application Number PCT/CN2020/118424, filed on Sep. 28, 2020, which claims priority of Chinese Patent Application Number 201910929614.0, filed on Sep. 29, 2019, the entire content of each of which is incorporated herein by reference.

### SEQUENCE LISTING

The instant application contains a Sequence Listing previously submitted to WIPO in ASCII format and the entire content of the electronic submission of the sequence listing is incorporated by reference in its entirety for all purposes. The ASCII file is named “221919_English_Translation_Sequence_listing rev.txt,” was last modified on Sep. 16, 2022, and is 81.093 bytes in size.

### TECHNICAL FIELD

Embodiments of the present disclosure concern biomedicine, and relates to anti-human Claudin18.2 antibodies or functional fragments thereof. Embodiments of the present disclosure also relate to the use of the antibodies or functional fragments thereof.

### BACKGROUND

Human tight junction protein Claudin18 (CLDN18) belongs to the family of tight junction proteins that biologically function in the construction of tight cell junctions, the maintenance of cell barrier functions and are involved in intercellular molecular trafficking. Claudin18 protein has a molecular weight of about 26 KD and can be changed by alternative splicing to Claudin subtypes having different properties: CLDN18.1 and CLDN18.2. CLDN18.1 and CLDN18.2 each comprises 261 amino acids and has four transmembrane domains, i.e., NH2 terminal domain and COOH terminal domain intracellularly located, and two extracellular loops (ECL1, ECL2). The extracellular regions 1 of CLDN18.1 and CLDN18.2 differ from each other by 8 amino acids.

CLDN18.2 is expressed in primary lesions and metastases of a number of human epithelial tumor types and is expressed continuously in diffuse carcinoma cells. Expression levels of CLDN18.2 are even more significantly increased in esophageal, pancreatic, lung and gastric cancer cells. A study by Sahin et al. suggested that CLDN18.2 is an important biomarker of gastric cancer, present in 70% of gastric cancer patients among which 90% to 95% were diagnosed with adenocarcinomas and another 5-10% comprised lymphomas, gastrointestinal stromal tumors (GISTs) and carcinoid tumors.

IMAB362, a monoclonal antibody against CLDN18.2, was developed by Ganymed Pharmaceuticals AG, a biotechnology company in Germany, and is currently in phase III clinical trials for the treatment of patients with advanced gastroesophageal cancer. The binding of the IMAB362 antibody alone leads to an inhibition of proliferation of target cells expressing CLDN18.2 both in vitro and in vivo, which inhibits tumor growth and eliminates the cancer cells via action mechanisms of Complement Dependent Cytotoxicity (CDC) and Antibody Dependent Cytotoxicity (ADCC), with various modes of action being shown to be independent but synergistic. The IMAB362 antibody is directed against indications including metastatic esophageal cancer, and metastatic gastric cancer, and is currently in phase III clinical trials in the US and the EU and is expected to file for marketing approval in 2021.

IMAB362 is a chimeric antibody, however, with relatively more murine amino acids and a relatively high probability of causing a rejection immune response in humans. Thus, there remains a need in the art for an antibody, in particular a humanized antibody, having a higher specificity, affinity and biological activity for human CLDN18.2. Furthermore, the two subtypes of Claudin18 have highly similar structures but different functions. The high homology between CLDN18.1 and CLDN18.2 and the higher structure of ECL1 make it more difficult to screen an antibody against CLDN18.2 of high quality, and a high degree of uncertainty with regard to antibody specificity exists.

### SUMMARY

According to embodiments of the present disclosure, an antibody capable of specifically binding to human CLDN18.2 with high affinity is obtained through hybridoma screening and humanization, by optimizing the preparation method of monoclonal antibodies and improving the screening efficiency of Claudin 18.2 specific antibodies. In some embodiments, the antibody has the fewest murine amino acids by humanization design and is expected to have better in vivo safety and application potential.

With respect to an immunogen, murine-derived cells expressing human CLDN18.2 are utilized in embodiments of the present disclosure. In one aspect, the murine-derived cells as host are non-immunogenic or have very low immunogenicity to mice, thereby avoiding the shadowing effect of the host cells on the immunogen of interest CLDN18.2 and avoiding the adverse effect on the production efficiency of CLDN18.2 specific hybridomas caused by introduction of a heterologous immunogen. In another aspect, expression of human CLDN18.2 on the surface of the cell membrane of murine-derived cells can be achieved, such that a monoclonal antibody obtained by the screening specifically binds to the extracellular region of CLDN18.2, ensuring the biological activity of the CLDN18.2 specific antibody.

With respect to screening strategy, a strategy which combines positive screening and negative screening is used according to embodiments of the present disclosure. Firstly, a positive screening is performed using recombinant cells expressing human CLDN18.2, to obtain monoclonal antibodies having high affinity to the cells; and then, a negative screening is performed using recombinant cells expressing human CLDN18.1 on the antibody clones capable of binding to recombinant cells expressing human CLDN18.2, to exclude monoclonal antibodies capable of specifically binding to a common extracellular region structure of CLDN18.2 and CLDN 18.1. Furthermore, the immunogen, the positive screening antigen, and the negative screening antigen used in embodiments of the present disclosure are all the same murine-derived cell platform, not only simplifying the process, avoiding the introduction of xenoantigens, but enabling the negative screening step to eliminate antibodies generated by the allogeneic antigen effect of murine derived cells on individual mice, and further improving the production efficiency of monoclonal antibodies specifically binding extracellular region 2 of CLDN18.2.

According to some embodiments of the present disclosure, an antibody or fragment thereof specifically binds human CLDN18.2. And some embodiments of the present disclosure are directed and to uses thereof. Fragments of the antibody according to embodiments of the present disclosure encompass, inter alia, various functional fragments of the antibody, such as antigen binding portions thereof, e.g. Fab, F(ab)₂ or scFv fragments.

Embodiments of the present disclosure provide at least the following technical solutions and/or advantages.

In one aspect, embodiments of the present disclosure provide a preparation method of an anti-human Claudin 18.2 antibody, the preparation method including:

(1) immunizing an animal with cells expressing human Claudin 18.2 protein on a surface as an immunogen;

(2) preparing cell clones capable of producing antibodies using the animals immunized in (1);

(3) using cells expressing human Claudin 18.2 protein on the surface as a positive screening antigen, screening antibodies having a binding activity to the positive screening antigen and cells producing the antibodies;

(4) using the cells expressing human Claudin 18.1 protein on the surface as a negative screening antigen, excluding antibodies having a binding activity to the negative screening antigen and cells producing the antibodies.

In preparation methods according to embodiments of the present disclosure, the cells expressing the human Claudin 18.2 protein in (1) are derived from the same species as the animal immunized. And in some embodiments, the cells expressing the human Claudin 18.2 protein are mouse cells, and the animal immunized is a mouse.

According to some embodiments, in the preparation methods according to embodiments of the present disclosure, the cell clones capable of producing antibodies in (2) are prepared by a technique selected from hybridoma techniques and single cell amplification techniques.

In some embodiments, in the preparation methods according to embodiments of the present disclosure, the positive screening antigen in (3) is the same as the immunogen in (1); and the negative screening antigen in (4) differs from the immunogen in (1) only in that the protein expressed is human Claudin 18.1 protein.

According to some embodiments, in the preparation methods according to embodiments of the present disclosure, (3) and (4) are each conducted by a method selected from Enzyme-Linked Immunosorbent Assay (ELISA) and Fluorescence Resonance Energy Transfer (FRET).

In another aspect, embodiments of the present disclosure provide an anti-human Claudin 18.2 antibody obtained according to the preparation methods described herein.

In some embodiments, the anti-human Claudin 18.2 antibody according to embodiments of the present disclosure specifically binds the N-terminus of human Claudin 18.2, for example, an extracellular region at the N-terminus of human Claudin 18.2 comprising the first Extracellular Loop (ECL1).

According to some embodiments, the anti-human Claudin 18.2 antibody according to embodiments of the present disclosure specifically has a specific binding to human CLDN18.2 at a nM scale; and has no significant difference in binding to human CLDN18.1 as compared to an isotype negative antibody (or an unrelated antibody).

In a further aspect, embodiments of the present disclosure provide an antibody or fragment thereof comprising a heavy chain variable region (VH) and a light chain variable region (VL), where the heavy chain variable region (VH) and the light chain variable region (VL) comprise a combination of CDRs (e.g., VH-CDR1, VH-CDR2, VH-CDR3; and VL-CDR1, VL-CDR2, VL-CDR3) selected from:

(1) VH-CDR1 as shown in SEQ ID NO. 31, VH-CDR2 as shown in SEQ ID NO. 32, and VH-CDR3 as shown in SEQ ID NO. 33; and VL-CDR1 as shown in SEQ ID NO. 34, VL-CDR2 as shown in SEQ ID NO. 35, and VL-CDR3 as shown in SEQ ID NO. 36;

(2) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 40, VL-CDR2 as shown in SEQ ID NO. 41, and VL-CDR3 as shown in SEQ ID NO. 42;

(3) VH-CDR1 as shown in SEQ ID NO. 43, VH-CDR2 as shown in SEQ ID NO. 44, and VH-CDR3 as shown in SEQ ID NO. 45; and VL-CDR1 as shown in SEQ ID NO. 46, VL-CDR2 as shown in SEQ ID NO. 41, and VL-CDR3 as shown in SEQ ID NO. 47;

(4) VH-CDR1 as shown in SEQ ID NO. 48, VH-CDR2 as shown in SEQ ID NO. 49, and VH-CDR3 as shown in SEQ ID NO. 50; and VL-CDR1 as shown in SEQ ID NO. 40, VL-CDR2 as shown in SEQ ID NO. 41, and VL-CDR3 as shown in SEQ ID NO. 51;

(5) VH-CDR1 as shown in SEQ ID NO. 52, VH-CDR2 as shown in SEQ ID NO. 53, and VH-CDR3 as shown in SEQ ID NO. 54; and VL-CDR1 as shown in SEQ ID NO. 55, VL-CDR2 as shown in SEQ ID NO. 41, and VL-CDR3 as shown in SEQ ID NO. 56;

(6) VH-CDR1 as shown in SEQ ID NO. 57, VH-CDR2 as shown in SEQ ID NO. 58, and VH-CDR3 as shown in SEQ ID NO. 33; and VL-CDR1 as shown in SEQ ID NO. 34, VL-CDR2 as shown in SEQ ID NO. 59, and VL-CDR3 as shown in SEQ ID NO. 60;

(7) VH-CDR1 as shown in SEQ ID NO. 61, VH-CDR2 as shown in SEQ ID NO. 62, and VH-CDR3 as shown in SEQ ID NO. 63; and VL-CDR1 as shown in SEQ ID NO. 46, VL-CDR2 as shown in SEQ ID NO. 41, and VL-CDR3 as shown in SEQ ID NO. 64;

(8) VH-CDR1 as shown in SEQ ID NO. 65, VH-CDR2 as shown in SEQ ID NO. 66, and VH-CDR3 as shown in SEQ ID NO. 67; and VL-CDR1 as shown in SEQ ID NO. 68, VL-CDR2 as shown in SEQ ID NO. 41, and VL-CDR3 as shown in SEQ ID NO. 69;

(9) VH-CDR1 as shown in SEQ ID NO. 65, VH-CDR2 as shown in SEQ ID NO. 70, and VH-CDR3 as shown in SEQ ID NO. 71; and VL-CDR1 as shown in SEQ ID NO. 72, VL-CDR2 as shown in SEQ ID NO. 41, and VL-CDR3 as shown in SEQ ID NO. 73;

(10) VH-CDR1 as shown in SEQ ID NO. 74, VH-CDR2 as shown in SEQ ID NO. 75, and VH-CDR3 as shown in SEQ ID NO. 76; and VL-CDR1 as shown in SEQ ID NO. 77, VL-CDR2 as shown in SEQ ID NO. 41, and VL-CDR3 as shown in SEQ ID NO. 78;

(11) VH-CDR1 as shown in SEQ ID NO. 79, VH-CDR2 as shown in SEQ ID NO. 80, and VH-CDR3 as shown in SEQ ID NO. 81; and VL-CDR1 as shown in SEQ ID NO. 82, VL-CDR2 as shown in SEQ ID NO. 41, and VL-CDR3 as shown in SEQ ID NO. 83;

(12) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 42;

(13) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 86;

(14) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 87;

(15) VH-CDR1 as shown in SEQ ID NO. 74, VH-CDR2 as shown in SEQ ID NO. 75, and VH-CDR3 as shown in SEQ ID NO. 76; and VL-CDR1 as shown in SEQ ID NO. 89, VL-CDR2 as shown in SEQ ID NO. 41, and VL-CDR3 as shown in SEQ ID NO. 90, and

(16) VH-CDR1 as shown in SEQ ID NO. 79, VH-CDR2 as shown in SEQ ID NO. 91, and VH-CDR3 as shown in SEQ ID NO. 81; and VL-CDR1 as shown in SEQ ID NO. 92, VL-CDR2 as shown in SEQ ID NO. 41, and VL-CDR3 as shown in SEQ ID NO. 93.

In some embodiments, in the antibody or fragment thereof according to embodiments of the present disclosure, the heavy chain variable region comprises an amino acid sequence as shown in any one of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 27, and SEQ ID NO. 29 or an amino acid sequence having at least 75% identity to the amino acid sequence as shown; and/or the light chain variable region comprises an amino acid sequence as shown in any one of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 25, SEQ ID NO. 26, SEQ ID NO. 28, and SEQ ID NO. 30 or an amino acid sequence having at least 75% identity to the amino acid sequence as shown.

According to some embodiments of the present disclosure, the antibody or fragment thereof comprises a heavy chain variable region and a light chain variable region selected from:

(1) an amino acid sequence as shown in SEQ ID NO. 1 or an amino acid sequence having at least 75% identity to the amino acid sequence as shown in SEQ ID NO. 1; and an amino acid sequence as shown in SEQ ID NO. 2 or an amino acid sequence having at least 75% identity to the amino acid sequence as shown in SEQ ID NO. 2;

(2) an amino acid sequence as shown in SEQ ID NO. 3 or an amino acid sequence having at least 75% identity to the amino acid sequence as shown in SEQ ID NO. 3; and an amino acid sequence as shown in SEQ ID NO. 4 or an amino acid sequence having at least 75% identity to the amino acid sequence as shown in SEQ ID NO. 4;

(3) an amino acid sequence as shown in SEQ ID NO. 5 or an amino acid sequence having at least 75% identity to the amino acid sequence as shown in SEQ ID NO. 5; and an amino acid sequence as shown in SEQ ID NO. 6 or an amino acid sequence having at least 75% identity to the amino acid sequence as shown in SEQ ID NO. 6;

(4) an amino acid sequence as shown in SEQ ID NO. 7 or an amino acid sequence having at least 75% identity to the amino acid sequence as shown in SEQ ID NO. 7; and an amino acid sequence as shown in SEQ ID NO. 8 or an amino acid sequence having at least 75% identity to the amino acid sequence as shown in SEQ ID NO. 8;

(5) an amino acid sequence as shown in SEQ ID NO. 9 or an amino acid sequence having at least 75% identity to the amino acid sequence as shown in SEQ ID NO. 9; and an amino acid sequence as shown in SEQ ID NO. 10 or an amino acid sequence having at least 75% identity to the amino acid sequence as shown in SEQ ID NO. 10;

(6) an amino acid sequence as shown in SEQ ID NO. 11 or an amino acid sequence having at least 75% identity to the amino acid sequence as shown in SEQ ID NO. 11; and an amino acid sequence as shown in SEQ ID NO. 12 or an amino acid sequence having at least 75% identity to the amino acid sequence as shown in SEQ ID NO. 12;

(7) an amino acid sequence as shown in SEQ ID NO. 13 or an amino acid sequence having at least 75% identity to the amino acid sequence as shown in SEQ ID NO. 13; and an amino acid sequence as shown in SEQ ID NO. 14 or an amino acid sequence having at least 75% identity to the amino acid sequence as shown in SEQ ID NO. 14;

(8) an amino acid sequence as shown in SEQ ID NO. 15 or an amino acid sequence having at least 75% identity to the amino acid sequence as shown in SEQ ID NO. 15; and an amino acid sequence as shown in SEQ ID NO. 16 or an amino acid sequence having at least 75% identity to the amino acid sequence as shown in SEQ ID NO. 16;

(9) an amino acid sequence as shown in SEQ ID NO. 17 or an amino acid sequence having at least 75% identity to the amino acid sequence as shown in SEQ ID NO. 17; and an amino acid sequence as shown in SEQ ID NO. 18 or an amino acid sequence having at least 75% identity to the amino acid sequence as shown in SEQ ID NO. 18;

(10) an amino acid sequence as shown in SEQ ID NO. 19 or an amino acid sequence having at least 75% identity to the amino acid sequence as shown in SEQ ID NO. 19; and an amino acid sequence as shown in SEQ ID NO. 20 or an amino acid sequence having at least 75% identity to the amino acid sequence as shown in SEQ ID NO. 20;

(11) an amino acid sequence as shown in SEQ ID NO. 21 or an amino acid sequence having at least 75% identity to the amino acid sequence as shown in SEQ ID NO. 21; and an amino acid sequence as shown in SEQ ID NO. 22 or an amino acid sequence having at least 75% identity to the amino acid sequence as shown in SEQ ID NO. 22;

(12) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 24 or an amino acid sequence having at least 75% identity to the amino acid sequence as shown in SEQ ID NO. 24;

(13) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 25 or an amino acid sequence having at least 75% identity to the amino acid sequence as shown in SEQ ID NO. 25;

(14) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 26 or an amino acid sequence having at least 75% identity to the amino acid sequence as shown in SEQ ID NO. 26;

(15) an amino acid sequence as shown in SEQ ID NO. 27 or an amino acid sequence having at least 75% identity to an amino acid sequence as set forth in SEQ ID NO. 27; and an amino acid sequence as shown in SEQ ID NO. 28 or an amino acid sequence having at least 75% identity to the amino acid sequence as shown in SEQ ID NO. 28;

(16) an amino acid sequence as shown in SEQ ID NO. 29 or an amino acid sequence having at least 75% of identity to the amino acid sequence as shown in SEQ ID NO. 29; and an amino acid sequence as shown in SEQ ID NO. 30 or an amino acid sequence having at least 75% of identity to the amino acid sequence as shown in SEQ ID NO. 30.

The at least 75% identity in the context of the present disclosure includes any percent identity greater than or equal to 75%, such as at least 80%, at least 85%, at least 90%, or at least 91%, 92%, 93%, 94%, 95%, 96%, 97%, 98%, or 99% identity.

The antibody or fragment thereof according to embodiments of the present disclosure may be in any form, e.g., a monoclonal antibody, a single chain antibody, a diabody, a single domain antibody, a nanobody, a fully or partially humanized antibody, or a chimeric antibody or the like. Alternatively, the antibody or fragment thereof may be a half-antibody or an antigen-binding fragment of the half-antibody, e.g., a single-chain variable fragment (scFv), bivalent single-chain variable fragment (BsFv), disulfide-stabilized Fv fragment (dsFv), (disulfide-stabilized Fv fragment)₂ (dsFv)₂, antigen-binding fragment (Fab), Fab′ fragment, F(ab′)₂ fragment, or variable fragment (Fv). With respect to the fragment provided by embodiments of the present disclosure, the antibody or fragment thereof may be any fragment of the antibody capable of binding to human Claudin 18.2.

In some embodiments, the antibody or fragment thereof may further comprise a human or murine constant region, for example a human or murine light chain constant region (CL) and/or heavy chain constant region (CH). In some embodiments, the antibody or fragment thereof may comprise a heavy chain constant region selected from IgG, IgA, IgM, IgD and IgE and/or a kappa or lambda type light chain constant region.

According to some embodiments, the antibody may be a monoclonal antibody, for example a murine, chimeric, or humanized monoclonal antibody. In some embodiments, the heavy chain constant region of the monoclonal antibody may be of a IgG1 or IgG4 subtype and the light chain constant region of the monoclonal antibody may be of a kappa type.

According to some embodiments of the present disclosure, the heavy chain constant region of the monoclonal antibody may comprise an amino acid sequence as shown in SEQ ID NO: 124 or an amino acid sequence having at least 75% identity to the amino acid sequence as shown. According to some embodiments of the present disclosure, the light chain constant region of the monoclonal antibody may comprise an amino acid sequence as shown in SEQ ID NO: 125 or an amino acid sequence having at least 75% identity to the amino acid sequence as shown.

In yet another aspect, embodiments of the present disclosure provide a nucleic acid molecule comprising a nucleotide sequence encoding the antibody or fragment thereof according to embodiments of the present disclosure, or encoding a heavy chain CDR, a light chain CDR, a heavy chain variable region, a light chain variable region, a heavy chain or a light chain comprised in the antibody or fragment thereof.

According to some embodiments of the present disclosure, the nucleic acid molecule may comprise a nucleotide sequence encoding the heavy chain variable region or the light chain variable region of the antibody according to embodiments of the present disclosure. For example, the nucleic acid molecule may comprise the nucleotide sequence as shown in any one of SEQ ID NOs. 96-125.

The nucleic acid molecule according to embodiments of the present disclosure can be cloned into a vector which in turn transfects or transforms a host cell. In yet another aspect, embodiments of the present disclosure provide a vector comprising the nucleic acid molecule according to embodiments of the present disclosure. The vector can be a eukaryotic expression vector, a prokaryotic expression vector, an artificial chromosome, a phage vector or the like.

The vector or nucleic acid molecule according to embodiments of the present disclosure may be used to transform or transfect a host cell or in any way enter a host cell for antibody preservation or expression, etc. Thus, in a further aspect, embodiments of the present disclosure provide a host cell comprising the nucleic acid molecule and/or vector according to embodiments of the present disclosure, or transformed or transfected with the nucleic acid molecule and/or vector according to embodiments of the present disclosure. The host cell may be any prokaryotic or eukaryotic cell, such as a bacterial or insect, fungus, plant or animal cell.

The antibody according to embodiments of the present disclosure can be obtained using any conventional techniques known in the art. For example, the heavy chain variable region and/or the light chain variable region of the antibody or the heavy chain and/or the light chain of the antibody may be obtained from the nucleic acid molecule provided by embodiments of the present disclosure, and then the antibody may be obtained by assembling them with optional other domains of the antibody. Alternatively, the host cell provided by embodiments of the present disclosure may be cultured under conditions that allow the host cell to express the heavy chain variable region and/or the light chain variable region of the antibody or the heavy chain and/or the light chain of the antibody may be assembled into an antibody. Optionally, the method may further include recovering the produced antibody.

The antibody or fragment thereof provided by embodiments of the present disclosure may also be combined with other moieties, for example, a cell surface receptor, a small molecule compound such as amino acids and carbohydrates, a small molecule polymer, or any other moiety that modifies the antibody of the present disclosure, or even an active protein or polypeptide. Thus, in another aspect, embodiments of the present disclosure provide a conjugate or fusion protein comprising an antibody or fragment thereof provided by embodiments of the present disclosure. For example, the conjugate or fusion protein can be a bispecific antibody comprising an antibody or fragment thereof according to embodiments of the present disclosure.

The antibody or fragment thereof, the nucleic acid molecule, the vector, the host cell, and/or the conjugate or fusion protein provided by embodiments of the present disclosure may be contained in a pharmaceutical composition, for example a pharmaceutical preparation, to be used for various purposes as needed. Thus, in a further aspect, embodiments of the present disclosure also provide a pharmaceutical composition comprising an antibody or fragment thereof, a nucleic acid molecule, a vector, a host cell, and/or a conjugate or fusion protein according to embodiments of the present disclosure, and optionally a pharmaceutically acceptable excipient.

For any purpose or use, embodiments of the present disclosure also provide a kit comprising an antibody or fragment thereof, a nucleic acid molecule, a vector, a host cell, a pharmaceutical composition and/or a conjugate or fusion protein according to embodiments of the present disclosure.

In another aspect, embodiments of the present disclosure also provide use of the antibody or fragment thereof, the nucleic acid molecule, the vector, the host cell, the pharmaceutical composition, and/or the conjugate or fusion protein as described herein in the manufacture of a medicament for the prevention and/or treatment of cancer. In some embodiments, the cancer may be selected from pancreatic cancer, gastric cancer, colon cancer, esophageal cancer, liver cancer, ovarian cancer, lung cancer, gallbladder cancer, and head and neck cancer.

In a further aspect, embodiments of the present disclosure also provide use of the antibody or fragment thereof, the nucleic acid molecule, the vector, the host cell, the pharmaceutical composition, and/or the conjugate or fusion protein as described herein in the manufacture of a reagent for the diagnosis of cancer. In some embodiments, the cancer may be selected from pancreatic cancer, gastric cancer, colon cancer, esophageal cancer, liver cancer, ovarian cancer, lung cancer, gallbladder cancer, and head and neck cancer.

In another aspect, embodiments of the present disclosure also provide a method of preventing and/or treating cancer, the method including administering to a subject in need thereof the antibody or fragment thereof, the nucleic acid molecule, the vector, the host cell, the pharmaceutical composition, and/or the conjugate or fusion protein according to embodiments of the present disclosure, and optionally other drug or means. The optional other drug or means refers to other drugs or means that can be administered in combination with the antibody or fragment thereof, the nucleic acid molecule, the vector, the host cell, the pharmaceutical composition, and/or the conjugate or fusion protein according to embodiments of the present disclosure, such as a small molecule drug, a targeted drug, a recombinant protein drug such as an antibody, a vaccine, an ADC, an oncolytic virus, a gene or nucleic acid therapy drug and/or radiotherapy. The co-administration of the two may be in any manner, including simultaneously, sequentially or at intervals.

In some embodiments, the cancer may be selected from pancreatic cancer, gastric cancer, colon cancer, esophageal cancer, liver cancer, ovarian cancer, lung cancer, gallbladder cancer, and head and neck cancer. According to some embodiments, the subject may be a mammal, for example, the subject may be a human.

Alternatively, embodiments of the present disclosure also provide a method for diagnosing cancer, the method including contacting the antibody or fragment thereof, the nucleic acid molecule, the vector, the host cell, the pharmaceutical composition, and/or the conjugate or fusion protein according to embodiments the present disclosure with a sample from a subject. In some embodiments, the cancer may be selected from pancreatic cancer, stomach cancer, colon cancer, esophageal cancer, liver cancer, ovarian cancer, lung cancer, gallbladder cancer, and head and neck cancer. According to some embodiments, the subject may be a mammal, for example, the subject may be a human.

The genes encoding the anti-human Claudin 18.2 antibody according to embodiments of the present disclosure were transduced onto primary T cells from a healthy donor through a lentiviral vector, and the prepared CAR-T cells were found to be efficiently activated by cells expressing human Claudin 18.2. Thus, in a further aspect, embodiments of the present disclosure also provide the use of the antibody or fragment thereof, the nucleic acid molecule, the vector, the host cell, the pharmaceutical composition, and/or the conjugate or fusion protein according to embodiments of the present disclosure in the manufacture of CAR-T cells.

Embodiments of the present disclosure provide an antibody capable of specifically binding to human Claudin 18.2. In contrast to existing anti-human Claudin 18.2 antibodies, the antibodies according to embodiments of the present disclosure have at least the following characteristics.

The anti-human Claudin 18.2 antibodies provided by embodiments of the present disclosure exhibit a strong affinity for the antigen Claudin 18.2, and have significant Complement Dependent Cytotoxicity (CDC) activity and antibody dependent cytotoxicity (ADCC) activity on target expressing cells, both of which are stronger than or comparable to IMAB362. Furthermore, the anti-human Claudin 18.2 antibodies according to embodiments of the present disclosure exhibit a higher specificity to human CLDN18.2 as compared to IMAB362.

In addition, the genes encoding the anti-human Claudin 18.2 antibody according to embodiments of the present disclosure were transduced onto primary T cells from a healthy donor through a lentiviral vector, and CAR-T cells were prepared. Upon the stimulation of target cells, the prepared CAR-T cells were detected for activating protein CD25 on the surface of CAR-T cells and for the secreted IFNγ, and were found to be efficiently activated by cells expressing human Claudin 18.2, demonstrating the target specificity of the antibodies according to embodiments of the present disclosure for human CLDN 18.2.

### Definitions

Unless otherwise defined, the meanings of scientific and technical terms used herein are the meanings commonly understood by those skilled in the art. The nomenclatures and techniques used in cell and tissue culture, molecular biology, and protein and oligo or polynucleotide chemistry and hybridization described herein are well known and commonly used in the art. Standard techniques are used for recombinant DNA, oligonucleotide synthesis, and tissue culture and transformation (e.g., electroporation, lipid transfection). Enzymatic reaction and purification techniques are performed according to the manufacturer's specifications or procedures commonly used in the art or described herein. These techniques and procedures are used as described in the many comprehensive and more specific literatures commonly known in the art and cited and discussed in this specification. See, e.g., Sambrook et al, Molecular Cloning: A Laboratory Manual (2nd ed., Cold Spring Harbor Laboratory Press, Cold Spring Harbor, N.Y. (1989)). The nomenclatures and laboratory procedures and techniques used in analytical chemistry, synthetic organic chemistry, and medical and pharmaceutical chemistry described herein are well known and commonly used in the art.

As used herein, the term “antibody” refers to an immunoglobulin molecule that generally consists of two pairs of polypeptide chains (each pair with one “light” (L) chain and one “heavy” (H) chain). In a general sense, the heavy chain can be interpreted as a polypeptide chain with a larger molecular weight in an antibody, and the light chain refers to a polypeptide chain with a smaller molecular weight in an antibody. Light chains are classified as κ and λ, light chains. Heavy chains are generally classified as μ, δ, γ, α, or ε, and isotypes of antibodies are defined as IgM, IgD, IgG, IgA, and IgE, respectively. In light chains and heavy chains, the variable region and constant region are linked by a “J” region of about 12 or more amino acids, and the heavy chain also comprises a “D” region of about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant region of the antibody can mediate the binding of immunoglobulins to host tissues or factors, including the binding of various cells of the immune system (e.g., effector cells) to the first component (C1q) of the classical complement system. The VH and VL regions can be further subdivided into highly variable regions (called complementarity determining regions (CDRs)), and between which conservative regions called framework regions (FRs) are distributed. Each VH and VL consists of 3 CDRs and 4 FRs arranged from amino terminus to carboxyl terminus in the following order: FR1, CDR1, FR2, CDR2, FR3, CDR3, FR4. The variable regions (VH and VL) of each heavy chain/light chain pair form an antibody binding site, respectively. The assignment of amino acids to each region or domain follows the definition of Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883. In particular, the heavy chain may also comprise more than 3 CDRs, such as 6, 9, or 12. For example, in the bispecific antibodies according to embodiments of the present disclosure, the heavy chain may be a ScFv with the N-terminus of the heavy chain of IgG antibody linked to another antibody, and in this case, the heavy chain comprises 9 CDRs.

As used herein, the term “antigen binding fragment” refers to the polypeptide comprising the fragment of a full-length antibody, which maintains the ability to specifically bind to the same antigen to which the full-length antibody binds, and/or competing with the full-length antibody for the specific binding to an antigen, which is also known as the “antigen binding portion”. See generally, Fundamental Immunology, Ch. 7 (Paul, W., ed., 2nd edition, Raven Press, N.Y. (1989), which is incorporated herein by reference in its entirety for all purposes. An antigen binding fragment of the antibody can be produced by recombinant DNA technology or by enzymatic or chemical cleavage of intact antibodies. In some cases, the antigen binding fragment includes a Fab, a Fab′, an F(ab′)2, an Fd, an Fv, a dAb, a complementarity determining region (CDR) fragment, a single chain antibody fragment (e.g., scFv), a chimeric antibody, a diabody and such polypeptide, which comprises at least a portion of the antibody sufficient to impart specific antigen binding ability to a polypeptide.

As used herein, the term “Fv fragment” refers to an antibody fragment consisting of the VL and VH domains of a single arm of an antibody. The term “Fab fragment” refers to an antibody fragment consisting of VL, VH, CL, and CH1 (or CH) domains. And the term “F(ab′)2” refers to an antibody fragment comprising two Fab fragments linked by the disulfide bridge on a hinge region.

In some cases, the antigen binding fragment of the antibody may be a single chain binding fragment (e.g., scFv) in which the VL and VH domains are paired to form a monovalent molecule via a linker that enables them to produce a single polypeptide chain (see, e.g., Bird et al., Science 242:423-426 (1988) and Huston et al., Proc. Natl. Acad. Sci. USA 85:5879-5883 (1988)). Such scFv molecules may have a general structure: NH2-VL-linker-VH—COOH or NH2-VH-linker-VL-COOH. An appropriate linker consists of a repeating G4S amino acid sequence or a variant thereof. For example, a linker having the amino acid sequence (G4S)4 can be used, but variants thereof can also be used (Holliger et al. (1993), Proc. Natl. Acad. Sci. USA 90: 6444-6448).

Antigen binding fragments (e.g., the above-mentioned antibody fragments) of antibodies can be obtained from given antibodies by using conventional techniques known to those skilled in the art (e.g., recombinant DNA technology or enzymatic or chemical cleavage), and the antigen binding fragments of the antibodies are screened for specificity in the same way as for intact antibodies.

As used herein, unless otherwise clearly defined in the context, when referring to the term “antibody”, it includes not only intact antibodies but also antigen binding fragments of antibodies.

As used herein, the term “isolated” refers to a state obtained from the natural state by artificial means. If a certain “isolated” substance or component is present in nature, it is possible because its natural environment changes, or the substance is isolated from the natural environment, or both. For example, a certain unisolated polynucleotide or polypeptide naturally exists in a certain living animal body, and the same polynucleotide or polypeptide with high purity may be isolated from such a natural state and thus called an isolated polynucleotide or polypeptide. The term “isolated” does not exclude mixed artificial or synthesized substances or other unpure substances that do not affect the activity of the isolated sub stance.

The term “host cell” refers to a cell into which a vector can be introduced, including but not limited to prokaryotic cells such as Escherichia coli cells, fungal cells such as yeast cells, insect cells such as Drosophila melanogaster (S2) cells or Spodoptera frugiperda (Sf9) cells, or animal cells such as fibroblasts, CHO cells, COS cells, NSO cells, HeLa cells, BHK cells, HEK293 cells or human cells, etc.

The term “KD” refers to the equilibrium dissociation constant (KD) of a specific antibody-antigen interaction, which is used to describe the binding affinity between the antibody and the antigen. The smaller the equilibrium dissociation constant, the closer the antibody-antigen binding, and the higher the affinity between the antibody and the antigen. In general, an antibody binds to an antigen at an equilibrium dissociation constant less than about 10⁻⁵ M, for example, less than about 10⁻⁶ M, 10⁻⁷ M, 10⁻⁸ M, 10⁻⁹ M or 10⁻¹⁰ M or less, for example, as determined by surface plasmon resonance (SPR) in a BIACORE instrument. For example, the affinity of an antibody with a cell is detected on a KINEXA 400 instrument using KINEXA method.

The term “Specific binding” means that an antibody reacts with one or more antigenic determinants of an antigen but does not react with other polypeptides, or it binds to other polypeptides with very low affinity (Kd>10⁻⁶). The antibody may include but is not limited to polyclonal, monoclonal, chimeric, dAb (domain antibody), single chain, Fab, Fab′ and F(ab′)2 fragment, Fv, scFv and Fab expression library. A monoclonal antibody (mAb) is an antibody obtained from a monoclonal cell line, and the cell line is not limited to eukaryotic, prokaryotic or phage clonal cell lines. A monoclonal antibody or antigen-binding fragment thereof can be obtained by recombination using, for example, hybridoma technology, recombination technology, phage display technology, and synthesis techniques such as CDR grafting, or other known techniques.

The “murine antibody” according to embodiments of the present disclosure is a monoclonal antibody against human CLDN18.2 produced according to the knowledge and skill in the art. During the production, the test subject is injected with the CLDN18.2 antigen, and then the hybridomas expressing the antibody with the desired sequence or functional property are isolated.

The “chimeric antibody” according to embodiments of the present disclosure is an antibody formed by fusing the variable regions of a murine-derived antibody with the constant regions of a human antibody, which can reduce the immune response induced by the murine-derived antibody. To establish a chimeric antibody, it is necessary to establish hybridoma secreting murine-derived specific monoclonal antibodies first, clone the variable region genes from the mouse hybridoma cells, and then clone the constant region genes of the human antibody as needed, and chimeric genes formed by linking the mouse variable region with the human constant region genes are inserted into an expression vector. Finally, the chimeric antibody is expressed in a eukaryotic system or a prokaryotic system.

The “humanized antibody” according to embodiments of the present disclosure is also called a CDR grafted antibody, which is the antibody produced by grafting mouse CDR sequences into the variable region framework (FR) of a human antibody. Such variable region framework sequences can be obtained from public DNA databases or public references, for example, from the ImMunoGeneTics (IMGT) website http://imgt.cines.fr or from the Journal of Immunoglobulin, 2001ISBN012441351.

The term “CLDN18.2” includes isoforms, mammalian (e.g. human) CLDN18.2, species homologs of human CLDN18.2, and analogs having at least one epitope common with human CLDN18.2. The amino acid sequence of CLDN18.2 (e.g. human CLDN18.2) is known in the art, for example as shown in the NCBI database.

The term “CLDN18.1” includes isoforms, mammalian (e.g. human) CLDN18.1, species homologs of human CLDN18.1, and analogs having at least one epitope common with human CLDN18.1. The amino acid sequence of CLDN18.1 (e.g. human CLDN18.1) is known in the art, for example as shown in the NCBI database.

“Optional”, “optionally”, “any”, or “any one” means that the following event or situation may, but does not necessarily occur, and the description includes the instances in which the event or situation does or does not occur. For example, “optionally contains 1 antibody heavy chain variable region” means the antibody heavy chain variable region with a specific sequence may be, but is not necessarily, present.

The term “pharmaceutical composition” refers to a mixture containing one or more compounds according to embodiments of the present disclosure or a physiologically/pharmaceutically acceptable salt or prodrug thereof together with other chemical components, as well as other components such as a physiologically/pharmaceutically acceptable carrier and/or excipient.

The pharmaceutical composition is used to facilitate administration to an organism, and facilitate absorption of the active ingredient to exert biological activities. A pharmaceutical (or therapeutic) composition should generally be sterile and stable under conditions of manufacture and storage. The composition can be formulated as a solution, a microemulsion, a dispersion, a liposome or other ordered structure suitable for a high antibody concentration. The pharmaceutical composition may be prepared as a sterile injectable solution by incorporating the active compound (i.e., the antibody or antibody portion according to embodiments of the present disclosure) in a required amount in an appropriate solvent with one ingredient or a combination of ingredients as listed above, and as required followed by being filtered for sterilization.

The methods, compositions, and combination therapies according to embodiments of the present disclosure can be combined with other active agents or therapeutic methods. The method may include the administration of the CLDN18.2 antibody molecule according to embodiments of the present disclosure to a subject in an amount effective for treatment or prevention of a disease (e.g., a cancer), optionally, in combination with one or more inhibitors, e.g., selected from PD-1, PD-L1, PD-L2, LAG-3, CTLA-4, or Tim-3 antibodies (immunotherapy) or other tumor therapeutic antibodies, such as Her-2, EGFR, VEGF, VEGFR antibodies, etc., as well as ADC (antibody drug conjugate, such as T-DM1), bispecific antibodies, chemotherapy drugs, etc. The method may also include administration of the CLDN18.2 antibody molecule as well as additional active agents, and the CLDN18.2 antibody molecule, additional active agents or all can be administered in such amount or dose that is higher, lower, or equal to the amount or dose of each active agent used alone (e.g., as a monotherapy). The administered amount or dose of the CLDN18.2 antibody molecule, the additional active agents or all of them may be lower (e.g., at least 20%, at least 30%, at least 40%, or at least 50%) than the amount or dose of each active agent used alone (e.g., as a monotherapy).

Furthermore, the anti-CLDN 18.2 antibody according to embodiments of the present disclosure can bind to CLDN18.2 to induce apoptosis of target cells (tumor cells), inhibit growth of tumor cells, increase ADCC against tumor cells by effector cells in vivo, and CDC killing effect for treating cancer patients. Accordingly, in certain embodiments, the anti-CLDN 18.2 antibody molecule described herein may exhibit an anti-tumor effect of the antibody according to embodiments of the present disclosure through these mechanisms. And a method of inhibiting growth of tumor cells is provided in some embodiments, which includes administering a therapeutically effective amount of the anti-CLDN 18.2 antibody molecule described herein to a subject. The method is suitable for in vivo treatment of cancer. To obtain a target specific therapeutic effect, the anti-CLDN 18.2 antibody molecule may be administered together with other antibodies. When the CLDN18.2 antibody is administered in combination with one or more active agents, the combination may be administered to a cancer type, for example, to a patient having a CLDN18.2 expressing tumor, in any order or simultaneously. In certain aspects, a method of treating (e.g., reducing or alleviating) a hyperproliferative condition or disease (e.g., cancer)—such as a solid tumor, hematological cancer, soft tissue tumor or metastatic lesion—in a subject is provided. The method includes administering one or more anti-CLDN 18.2 antibody molecules described herein to a subject alone or in combination with other active agents or treatments.

As used herein, the terms “carcinoma”, “cancer”, “cancer patient”, and the like are intended to include all types of cancerous growth or tumorigenic processes, metastatic tissues or malignant transformed cells, tissues or organs, regardless of the histopathological type or aggressive stage. Examples include but are not limited to solid tumors, hematological cancers, soft tissue tumors, and metastatic lesions. Nonlimiting examples of solid tumors include malignancies, e.g. sarcomas and carcinomas of multiple organ systems (including adenocarcinomas and squamous cell carcinomas), such as those invading the liver, lung, breast, lymph, gastrointestinal tract (e.g. colon), genitourinary tract (e.g. kidney, bladder epithelial cells), prostate and pharynx. Nonlimiting examples of adenocarcinomas include malignancies such as non-small cell carcinomas in the majority of colon, rectal, stomach, renal cell, liver, and lung cancers, small intestine cancer and esophagus cancer. Nonlimiting examples of squamous cell carcinomas include malignancies, such as in the lung, esophagus, skin, head and neck region, oral cavity, anus and cervix. Metastatic lesions of the aforementioned cancers may also be treated or prevented using the methods and compositions according to embodiments of the present disclosure. The antibody molecules directed to CLDN18.2 may be combined with an immunogenic agent such as cancer cells, purified tumor antigens (including recombinant proteins, peptides and sugar molecules), cells and cells transfected with a gene encoding an immunostimulatory cytokine.

As used herein, term “antibody-dependent cell-mediated cytotoxicity” (ADCC) describes the cell killing ability of effector cells (in particular lymphocytes) which may require the target cell being marked by an antibody. ADCC preferably occurs when antibodies bind to antigens on tumor cells and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed T-cell responses. In some embodiments, in vivo induction of ADCC will lead to tumor-directed T-cell responses and host-derived antibody responses.

As used herein, the term “Complement-dependent cytotoxicity” (CDC) is another cell killing method that can be directed by antibodies. IgM is the most effective isotype for complement activation. IgG1 and IgG3 are also both very effective at directing CDC via the classical complement activation pathway. In some embodiments, in this cascade, the formation of antigen-antibody complexes results in the uncloaking of multiple C1q binding sites in close proximity on the CH2 domains of participating antibody molecules such as IgG molecules (C1q is one of three subcomponents of complement CI). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. In some embodiments, the complement cascade ends in the formation of a membrane attack complex, which creates pores in the cell membrane that facilitate free passage of water and solutes into and out of the cell.

It has been surprisingly found that the antibody-drug conjugates prepared with the anti-CLDN 18.2 antibodies described herein are capable of mediating the killing of cells, in particular of cells expressing CLDN18.2, such as cancer cells, by inducing Complement Dependent Cytotoxicity (CDC) mediated lysis and/or Antibody Dependent Cellular Cytotoxicity (ADCC) mediated lysis. Accordingly, in some embodiments, the antibody-drug conjugates according to embodiments of the present disclosure mediate cell killing by inducing Complement Dependent Cytotoxicity (CDC) mediated lysis and/or Antibody Dependent Cellular Cytotoxicity (ADCC) mediated lysis, for example by inducing CDC mediated lysis and ADCC mediated lysis.

### BRIEF DESCRIPTION OF THE DRAWINGS

Embodiments of the present disclosure are described in detail below with reference to the attached figures, in which:

FIG. 1 shows the results of an in vitro cell binding assay of anti-human CLDN18.2 chimeric antibodies.

FIG. 2 shows the results of a Complement Dependent Cytotoxicity (CDC) assay of anti-human CLDN18.2 humanized antibodies, in which panel 2A: CHOK1; panel 2B: BxPC3; panel 2C: NCI-N87.

FIG. 3 shows the results of an Antibody Dependent Cellular Cytotoxicity (ADCC) assay of anti-human CLDN18.2 humanized antibodies, in which panel 3A: CHOK1; panel 3B: BxPC3; panel 3C: NCI-N87.

FIG. 4 shows the result of analysis of the binding characteristics of the anti-human CLDN18.2 humanized antibodies to proteins of the same family.

FIG. 5 shows the binding of anti-human CLDN18.2 humanized antibodies at different concentrations to human CLDN18.1 expressing cells.

FIG. 6 shows the results of analysis of species cross binding characteristics of anti-human CLDN18.2 humanized antibodies.

FIG. 7 shows the results of detected positive percentages of CAR-T cells prepared in different groups.

FIG. 8 shows different antigen expression profiles of different CHO cell strains constructed.

FIG. 9 shows the results of detected IFN-γ in supernatants after the incubation of CAR-T cells in different groups with target cells.

FIG. 10 shows the results of detected CD25 expression on CAR-T cells in different groups after incubation with target cells.

### DETAILED DESCRIPTION

Embodiments of the present disclosure are illustrated below with reference to specific examples. It will be understood by those skilled in the art that these examples are merely illustrative of the present disclosure and do not limit the scope of the present disclosure in any way.

The experimental procedures in the following examples are all conventional, unless otherwise specified. The raw materials and reagents used in the following examples are all commercially available products, unless otherwise specified.

The heavy and light chain sequences of IMAB362 are as shown in SEQ ID NO. 126 and SEQ ID NO. 127.

Antigen human CLDN18.2 is as shown in NP 001002026.1, and antigen human CLDN18.1 is as shown in NP 057453.1.

The negative control (Isotype control) in the figures is anti-CD33 IgG antibody Lintuzumab in full length.

### Example 1 Screening for Murine Monoclonal Antibodies

Balb/c mice were immunized with CHOK1 cells stably expressing human CLDN18.2 protein on cell surfaces. One month later, sera from the mice were analyzed by flow cytometry (FACS), and spleens were taken from mice having high antibody titers in sera. The spleen cells isolated by standard methods were fused with myeloma cells P3X63Ag8.653 using PEG or electrofusion methods. The fused hybridoma cells were seeded in 384 well plates, and after 10-14 days of culture, supernatants obtained were analyzed by FACS for antibody secretion by the hybridoma cells. Several clones were obtained that were able to bind to CHOK1 cells stably expressing human CLDN18.2 protein on cell surfaces and were not able to bind to CHOK1 cells stably expressing human CLDN18.1 protein on cell surfaces. Single cells of the obtained clones were obtained by Limiting Dilution, and each of the monoclonal hybridoma cell clones obtained after being diluted three times secreted only one antibody.

The monoclonal hybridoma cells secreting anti human CLDN18.2 were subjected to expansion culture, and the total RNA of the cells was extracted using RNAfast200 Kit (Shanghai Flytech Biotechnology Co., Ltd.) according to the steps described in the Kit instructions. The total RNA of the hybridoma cells obtained was reverse transcribed to cDNA using 5 xPrimeScript RT Master Mix (Takara). And sequences of antibody light chain variable region IgVL (κ) and heavy chain variable region VH were amplified using degenerate primers (Anke Krebber., 1997) and Extaq PCR reagents (Takara). PCR amplification products were purified using PCR Clean-up Gel Extraction Kit (Macherey-Nagel GmbH & Co.), linked to T-vector using pClone007 Simple Vector Kit (Tsingke Biotechnology Co., Ltd.) according to Kit instructions, and transformed into competent Escherichia coli cells. Variable region sequences of the monoclonal antibodies were obtained by DNA sequencing after strain amplification and plasmid extraction.

TABLE 1
Light and heavy chain variable regions of murine antibodies

| Murine antibody | Heavy chain variable region (VH) | Light chain variable region (VL) |
|---|---|---|
| 11M23 | SEQ ID NO: 1 | SEQ ID NO: 2 |
| 16K15 | SEQ ID NO: 3 | SEQ ID NO: 4 |
| 18B21 | SEQ ID NO: 5 | SEQ ID NO: 6 |
| 20L17 | SEQ ID NO: 7 | SEQ ID NO: 8 |
| 43B5 | SEQ ID NO: 9 | SEQ ID NO: 10 |
| 43L6 | SEQ ID NO: 11 | SEQ ID NO: 12 |
| 46J05 | SEQ ID NO: 13 | SEQ ID NO: 14 |
| 48G12 | SEQ ID NO: 15 | SEQ ID NO: 16 |
| 50C14 | SEQ ID NO: 17 | SEQ ID NO: 18 |
| 52E22 | SEQ ID NO: 19 | SEQ ID NO: 20 |
| 8K13 | SEQ ID NO: 21 | SEQ ID NO: 22 |

>11M23_vh (SEQ ID NO: 1)
QVLQKESGPDLVAPSQSLSITCTVSGFSLTNYGVHWVRQPPGKGLEWLVVIWSDGRINYNSALKSRLSITKDNSKRQVFLK
MNSLQIDDTAIYYCVRHPAFGPHAMDYWGQGISVTVSS
>11M23_vl (SEQ ID NO: 2)
DIVMTQDAPSIPVTPGESVSISCRSSKSLLNSNGNTYLYWFLQRPGQSPHLLLYRMSNPASGAPDRFSGSGSGTEFTLRIS
RVEAEDVGVYYCMQYLEYPPTFGAGTRLELK
>16K15_vl (SEQ ID NO: 3)
EVMLVESGGGLVRPGGSLKLSCAGSGITLSTYAMSWVRQTPERRLEWVASIISGGITYYLDSVKGRFTISRDNARNILYLQ
MSSLRSEDTAIYYCARKYHGNALDYWGQGTSVTVSL
>16K15_vl (SEQ ID NO: 4)
DIVMTQSPSSLPVTAGETVTMRCKSSQSLLNSGNQRNYLTWYQRKPGQPPKKLIYWASTRESGVPDRFTGSGSGTDFTLTI
SGVQAEDLAVYYCQNNYFYPLTFGAGTKLELK
>18B21_vh (SEQ ID NO: 5)
QIQMVQSGPELKKPGETVRISCKASGYSFTTAGMQWVRKMPGEGLKWIGWIIAHSGEPKYTEDFKGRFAFSLETSASTTYL
QISNLKNEDTATYFCARWGKGNTMDYWGQGTSVIVSS
>18B21_vl (SEQ ID NO: 6)
DIVMTQSPSSLTVTAGEKVTMSCKSSQSLLNGGNQRNYLTWYQQKPGQPPKLLIYWAST
RESGVPDRFTGSGSGTHFTLTISSVQAEDLAVYYCQNAYFFPLTFGAGTKLELK
>20L17_vh (SEQ ID NO: 7)
DVQLVESGGGLVQPGGSRKLSCAASGFTFSSFGMHWVRQAPEKGLEWVAYISSGSSTIYYPDTVKGRFTVSRDNPKNTLFL
QMTSLRSEDTAMYYCVRLGPRGNVMDHWGQGTSVTVSS
>20L17_vl (SEQ ID NO: 8)
DIVMTQSPSSLTVTAGEKVTMSCKSSQSLLNSGNQRNYLTWYQQKPGQPPKLLIYWASTRESGVPDRFTGSGSGTDFTLTI
SSVQAEDLAVYYCQNVYFYPLTFGTGTKLELR
>43B5_vh (SEQ ID NO: 9)
DVQLQESGPDLVKPSQSLSLTCTVSGYSISGAYNWHWIRQFPGNKLEWLAYMQYSGSSNYNPSFKSRISISRDTSKNQFFL
QLKSVTTEDTATYYCARMYNGNSFLYWGQGTLVTVSA
>43B5_vl (SEQ ID NO: 10)
DIVMTQSPSSLTVTAGEKVTMNCKSSQSLFNSGNQKNYLTWYQQKPGQPPRLLIYWASTRESGVPDRFTGSGSGTDFTLTI
SSVQAEDLSLYYCQNSYSYPLTFGAGTKLELK
>43L6_vh (SEQ ID NO: 11)
QVQLKESGPDLVAPSQSLSLTCSVSGFSLTSYGIHWVRQPPGKGLEWLVVIWSDGRTTYNSGLKSRLSISKDNSKSQVLLK
MNSLRTDDTAIYYCVRHPAFGPHAMDYWGQGTSVTVSS
>43L6_vl (SEQ ID NO: 12)
DIVMTQAAPSVPVTPGESVSISCRSSKSLLNSNGNTYLYWFLQRPGQSPQLLIYRMSNLASGVPDRFSGSGSGTDFTLRIS
RVEAGDVGVYYCMQYLEYPVTFGAGTKLELK
>46J05_vh (SEQ ID NO: 13)
DVQLVESGGGLVQPGGSRKLSCAASGFTFSRFGMHWVRQAPKKGLEWVAYISSGSNTIYYADTVKGRFTISRDNPKNTLFL
QTTSLRSEDTAIYYCGRLGFYGNSFDHWGQGTLVTVSA
>46J05_vl (SEQ ID NO: 14)
NILMTQSPSSLTVTAGEKVTMNCKSSQSLLNGGNQRNYLTWYQQKAGQPPKLLIYWASTRESGVPDRFTGGGSGTDFTLTI
SSVQAEDLALYYCQNSYYYPLTFGAGTKLELK
>48G12_vh (SEQ ID NO: 15)
EVQLRQSGPELVKPGASVKMSCKASGYTFTTYIINWVKQKPGQGLEWIGYINPYNDDTRYNERVKGKATLTSDKSSSTAYM
ELSSLTSEDSAVYYCARFYFGNSFTYWGQGTLVTVSA
>48G12_vl (SEQ ID NO: 16)
DIVMTQSPSSLPVTVGERVTMTCKSSQGLFNSGNQRNYLTWYQQKPGQPPKLLIYWASTRESGVPDRFTGSGSGTDFTLTI
SSVQAEDLAIYYCQNNYIYPLTFGAGTKLELK
>50C14_vh (SEQ ID NO: 17)
EVQLRQSGPELVKPGASVKMSCKASGYTFTTYIINWVKQKPGQGLEWIGYINPYNDGTRYNERVKGKATLTSDKSSSTAYM
ELSSLTSEDSAVYYCARFHFGNSFTYWGQGTLVTVSA
>50C14_vl (SEQ ID NO: 18)
DIVMTQSPSSLPVTTGEKVTMTCKSSQGLFNNGNQRNYLTWYQQKPGQPPKLLIYWASTRESGVPDRFIGSGSGTDFTLTI
SSVQAEDLAIYYCQNNYIFPLTFGAGTKLELK
>52E22_vh (SEQ ID NO: 19)
QIQLVQSGPELKKPGETVKISCKASGYTLTNYGMNWVRQAPGKGLKWMGWIRPNTGEPTYAEDFKGRFVFSLETSAATAYL
QITNLKSEDTSTYFCARLYRGNTLDNWGQGTSVIVSS
>52E22_vl (SEQ ID NO: 20)
DIVMTQSPSSLTVTTGEKVTMSCKSSQNLLNSGNQRNYLTWYQQKPGQSPKLLIYWASTRESGVPYRFTGSGSGTDFTLTI
SSVQTDDLAIYYCQNGYSFPFTFGSGTKLEIK
>8K13_vh (SEQ ID NO: 21)
QVHLQQSGAELVRPGSSVKISCKASGYAFSNYWMNWVRQRPGQGLEWIGQIYPGNGDTKYSGKFNSKDTLTADKSSNTAYM
QLNSLTSEDSAVYFCARFYYGNVMDYWGQGTSVTVSS
>8K13_vl (SEQ ID NO: 22)
DIVLTQSPSSLTVTAGEKVTMSCKSSQTLLNGGNQKNYLTWYQQKSGQPPKLLIYWASTRESGVPDRFTGSGSGTDFTLTI
SSVQAEDLAVYYCQNGYSYPLTFGVGTKLELK

### Example 2 Preparation of Anti-Human CLDN18.2 Chimeric Antibodies

The heavy chain variable region sequence of each murine anti-human CLDN18.2 monoclonal antibody and the heavy chain constant region sequence of the published human monoclonal antibody IgG1 subclass (see SEQ ID NO: 124) were spliced together and constructed into a mammalian cell expression vector. And the light chain variable region sequence of each murine anti-human CLDN18.2 monoclonal antibody and the light chain constant region sequence of the published human monoclonal antibody kappa subclass (see SEQ ID NO: 125) were spliced together and constructed into a mammalian cell expression vector. The constructed heavy chain and light chain vectors of the anti-human CLDN18.2 chimeric antibodies were mixed in pairs, HEK293 cells were transfected with the vectors using Polyethyleneimine (PEI), and cell supernatants were collected about 7 days later. Anti-human CLDN18.2 chimeric antibody proteins were obtained via Protein A purification.

The chimeric antibodies of embodiments of the present disclosure were named following a format “murine antibody abbreviation-xiIgG”.

### Example 3 Cell Binding Assay In Vitro of Anti-Human CLDN18.2 Chimeric Antibodies

The chimeric anti-human CLDN18.2 antibodies were diluted 2-fold in gradient from an initial concentration of 100 nM and solutions of each antibody of 16 concentrations were obtained. 10 μl of the solutions of each antibody of each concentration was added to a 384-well plate. CHOK1 cells expressing CLDN18.2 on the cell surface were collected by centrifugation for 5 min at 100 g at room temperature, and the cells were washed with PBS containing 0.5% BSA once and then were centrifuged for 5 min at 100 g at room temperature. The cells were resuspended at a density of about 2×10⁶ cells/ml, and 10 μl was added to each well of the 384-well plate to which the antibody had been added. After incubation for 1 hour at 4° C., fluorescently labeled secondary goat anti-human IgG antibody was added. After continued incubation for 1 hour at 4° C., the mean fluorescence readings of the cell populations were analyzed by a flow cytometer.

The results showed that the chimeric antibodies had specific binding to the cells expressing human CLDN18.2 at a nM scale. See FIG. 1.

### Example 4 Humanization of Anti-Human CLDN18.2 Murine Antibodies

Based on a comprehensive analysis of Kabat and Chothia antibody coding schemes, amino acid sequence regions of 6 complementarity-determining regions (CDRs) and framework regions supporting the conserved three-dimensional conformation of the heavy and light chains of each murine antibody were determined. Subsequently, the heavy chain variable region of the human antibody which mostly resembles the murine antibody, such as IGHV1|IGHJ4*01, was searched for and selected in known human antibody sequences. The framework region sequences were selected as a template, and the heavy chain CDRs of the murine antibody were combined with the framework regions of the human antibody, and a humanized heavy chain variable region sequence was ultimately produced. In the same manner, a humanized light chain variable region sequence was produced.

An Antibody with murine CDRs grafted directly to its human framework regions often exhibits a dramatic decrease in binding activity, thus requiring the conversion of individual amino acids in the framework regions from being human back to murine. In order to determine which positions need to be reverted to original murine residues, the designed humanized antibody sequence and the original murine antibody sequence should be compared to check for differences in the amino acids, and to check whether those different amino acids are important for supporting the antibody structure or for binding to the antigen. The sequences obtained by humanization design need to be checked for potential post-Translational Modification Sites (PTMs), such as an N (asparagine) glycosylation site, an N-deamidation site, a D (aspartic acid) isomerization site, etc.

The gene of each humanized heavy chain variable region sequence was constructed into a mammalian cell expression vector comprising the gene of the heavy chain constant region sequence of the human monoclonal antibody IgG1 subclass. And the gene of each humanized light chain variable region sequence was constructed into a mammalian cell expression vector comprising the gene of the light chain constant region sequence of the human monoclonal antibody kappa subclass. The constructed heavy chain and light chain vectors of the humanized anti-human CLDN18.2 antibodies were mixed in pairs, HEK293 cells were transfected with the vectors using Polyethyleneimine (PEI), and cell supernatants were collected about 7 days later. Anti-human CLDN18.2 humanized antibody proteins were obtained via Protein A purification.

Humanized antibodies of embodiments of the present disclosure were named following the format “murine antibody abbreviation-hz”. Antibodies having CDRs from murine antibodies grafted directly to their human framework regions were named following the format “murine antibody abbreviation-hz00”; and antibodies further engineered were numbered with the numbers of the humanized sequences.

The binding kinetic parameters of the chimeric antibodies and the humanized antibodies thereof to the antigen human CLDN18.2 were analyzed by a Fortebio (BLITZ pro1.1.0.28) instrument. Before performing the assay, an NTA bioprobe was soaked in PBS for 10 min, and the probe was then placed in PBS containing 100 nM antigen for 300 s to capture the His-tagged antigen. The probe was further subjected to a binding reaction with 100 nM antibody for a binding time of 400 s, and the probe was then transferred to PBS and subjected to a dissociation reaction for 600 s. When the assay was finished, data from which the response value of a blank control had been deducted were fitted to a 1:1 Langmuir binding model using software, and then the kinetic constants for antigen-antibody binding were calculated. The results are shown in Table 2.

TABLE 2
Comparison of binding kinetics parameters following
the humanization of murine antibodies

| | | | K_{D} | Kₒₙ | K_{off} | K_{off}/ |
|---|---|---|---|---|---|---|
| Ab ID | PTM | Response | (M) | (1/Ms) | (s⁻¹) | K_{off}(Xi-IgG) |
| 8K13-xiIgG | Yes | 0.134 | 3.82E−10 | 2.98E+05 | 1.14E−04 | 1.00 |
| 8K13-hz00 | Yes | 0.133 | 7.44E−10 | 2.66E+05 | 1.98E−04 | 1.74 |
| 8K13-hz11 | Yes | 0.139 | 2.97E−10 | 2.27E+05 | 6.73E−05 | 0.59 |
| 8K13-hz24 | No | 0.133 | 1.51E−09 | 2.64E+05 | 3.99E−04 | 3.50 |
| 11M23-xiIgG | Yes | 0.184 | 2.93E−10 | 3.66E+05 | 1.07E−04 | 1.00 |
| 11M23-hz00 | Yes | 0.164 | 1.49E−09 | 2.18E+05 | 3.24E−04 | 3.03 |
| 11M23-hz11 | Yes | 0.174 | 1.29E−09 | 3.56E+05 | 4.60E−04 | 4.30 |
| 11M23-hz22 | No | 0.136 | 2.92E−09 | 6.70E+05 | 1.95E−03 | 18.22 |
| 16K15-xiIgG | Yes | 0.261 | 5.57E−10 | 2.76E+05 | 1.54E−04 | 1.00 |
| 16K15-hz00 | Yes | 0.176 | 8.95E−11 | 2.40E+05 | 2.15E−05 | 0.14 |
| 16K15-hz11 | Yes | 0.17 | 5.35E−10 | 1.88E+05 | 1.00E−04 | 0.65 |
| 16K15-hz22 | No | 0.266 | 1.10E−09 | 2.59E+05 | 2.86E−04 | 1.86 |
| 52E22-xiIgG | Yes | 0.223 | 9.96E−11 | 3.57E+05 | 3.55E−05 | 1.00 |
| 52E22-hz00 | Yes | 0.188 | 3.09E−10 | 1.98E+05 | 6.11E−05 | 1.72 |
| 52E22-hz11 | Yes | 0.238 | 5.42E−10 | 3.63E+05 | 1.97E−04 | 5.55 |
| 52E22-hz12 | No | 0.226 | 3.60E−10 | 2.18E+05 | 7.83E−05 | 2.21 |

The results showed that the dissociation constant of 11M23-hz22 relative to the murine chimeric antibody was raised more than 10 fold after humanization and removal of the critical post translational modification site (PTM) and was therefore eliminated from the list of candidates. The dissociation constants of the remaining 8K13-hz24, 16K15-hz22 and 52E22-hz12 were raised 1 to 4 fold, compared to original murine chimeric antibodies and could be used as lead molecules for subsequent studies. The light chain amino acid sequence of 16K15-hz22 contained two consecutive “NN” residues, and further optimized mutant antibodies 16K15-hz22_2 and 16K15-hz22_3 were obtained by changing the “NN” residues to “SN” and “QN” residues, respectively.

TABLE 3
Light and heavy chain variable regions of humanized antibodies

| | Humanized light chain | Humanized heavy chain | Humanized |
|---|---|---|---|
| Murine antibody | variable region | variable region | antibody |
| 16K15 | SEQ ID NO. 128 | SEQ ID NO. 129 | 16K15-hz00 |
| | SEQ ID NO. 128, having | SEQ ID NO. 129, having | 16K15-hz11 |
| | reverse mutations at | reverse mutation at position 24 | |
| | positions 52 and 89 | | |
| | SEQ ID NO. 24 | SEQ ID NO. 23 | 16K15-hz22 |
| | SEQ ID NO. 25 | | 16K15-hz22_2 |
| | SEQ ID NO. 26 | | 16K15-hz22_3 |
| 52E22 | SEQ ID NO. 130 | SEQ ID NO. 131 | 52E22-hz00 |
| | SEQ ID NO. 130, having | SEQ ID NO. 27 | 52E22-hz11 |
| | reverse mutation at | | |
| | position 89 | | |
| | SEQ ID NO. 28 | SEQ ID NO. 27 | 52E22-hz12 |
| 8K13 | SEQ ID NO. 132 | SEQ ID NO. 133 | 8K13-hz00 |
| | SEQ ID NO. 132, having | SEQ ID NO. 133, having | 8K13-hz11 |
| | reverse mutations at | reverse mutations at positions | |
| | positions 4 and 89 | 74 and 77 | |
| | SEQ ID NO. 30 | SEQ ID NO. 29 | 8K13-hz24 |
| 11M23 | SEQ ID NO. 88 | SEQ ID NO. 84 | 11M23-hz00 |
| | SEQ ID NO. 88, having | SEQ ID NO. 84, having | 11M23-hz11 |
| | reverse mutation at | reverse mutation at position | |
| | position 41 | 71 | |
| | SEQ ID NO. 88, having | SEQ ID NO. 84, having | 11M23-hz22 |
| | reverse mutation at | reverse mutation at position | |
| | position 41 as well as | 71 as well as D54E and N60Q | |
| | N31S and N33S | | |

>16K15_vl_hz2 (SEQ ID NO: 24)
DIVMTQSPDSLAVSLGERATINCKSSQSLLSSGNQRNYLTWYQQKPGQPPKKLIYWASTRESGVPDRFSGSGSGTDFTLTISS
LQAEDLAVYYCQNNYRYPLTFGQGTKLEIK
>16K15_vl_hz2_N-S (SEQ ID NO: 25)
DIVMTQSPDSLAVSLGERATINCKSSQSLLSSGNQRNYLTWYQQKPGQPPKKLIYWASTRESGVPDRFSGSGSGTDFTLTISS
LQAEDLAVYYCQSNYFYPLTFGQGTKLEIK
>16K15_vl_hz2_N-Q (SEQ ID NO: 26)
DIVMTQSPDSLAVSLGERATINCKSSQSLLSSGNQRNYLTWYQQKPGQPPKKLIYWASTRESGVPDRFSGSGSGTDFTLTISS
LQAEDLAVYYCQQNYFYPLTFGQGTKLEIK
>16k15_vh_hz2 (SEQ ID NO: 23)
EVQLVESGGGLVQPGGSLRLSCAGSGITLSTYAMSWVRQAPGKGLEWVSSIISGGITYYLDSVKGRFTISRDNAKNTLYLQMN
SLRAEDTAVYYCARKYHGNALDYWGQGLTVTVSS
>52E22_vl_hz2 (SEQ ID NO: 28)
DIVMTQSPDSLAVSLGERATINCKSSQNLLSSGNQRNYLTWYQQKPGQPPKLLIYWASTRESGVPDRFSGSGSGTDFTLTISS
LQAEDLAVYYCQQGYSFPFTFGQGTKLEIK
>52E22_vh_hz1 (SEQ ID NO: 27)
QIQLVQSGSELKKPGASVKVSCKASGYTLTNYGMNWVRQAPGQGLEWMGWIRPNTGEPTYAEDFKGRFVFSLDTSVATAYLQI
TSLKAEDTAVYYCARLYRGNTLDNWGQGTLVTVSS
>8K13_vl_hz4_N-S_N-Q (SEQ ID NO: 30)
DIVLTQSPDSLAVSLGERATINCKSSQTLLSGGNQKNYLTWYQQKPGQPPKLLIYWASTRESGVPDRFSGSGSGTDFTLTISS
LQAEDLAVYYCQQGYSYPLTFGQGTKLEIK
>8K13_vh_hz2 (SEQ ID NO: 29)
QVQLVQSGAEVKKPGSSVKVSCKASGYAFSNYWMNWVRQAPGQGLEWMGQIYPGSGDTKYSGKFQSRVTITADKSTNTAYMEL
SSLRSEDTAVYYCARFYYGNVMDYWGQGTLVTVSS

### Example 5 Kinetic Experiment of Binding Affinity In Vitro of Anti-Human CLDN18.2 Humanized Antibodies

Antibody-antigen interactions were measured using BIAcore 5200 from GE Healthcare.

Referring to the instructions provided in the Human Antibody Capture Kit (cat No. BR-1008-39, Lot 10261753) from GE Healthcare, the analytical channel and the control sample channel on a CM5 sensor chip were first saturated and coupled with the maximum amount of anti-human Fc antibody, then buffers containing anti-human CLDN18.2 chimeric antibodies, humanized antibodies or a control antibody IMAB362 at 7.5 μg/ml were allowed to flow through the analytical channel and uniformly distribute. Finally, antigen samples diluted in gradient (the initial concentration was 20 nM and diluted by 1:3 to get 8 concentrations, and the concentration 0.741 nM was set to be repeated) were allowed to flow through both the analytical channel and the sample channel, and the photoreactions upon the binding of antibody to antigen were measured. Subsequently, the association constant Kon and dissociation constant Koff and affinity constant KD of each antibody were finally obtained by instrument software fitting analysis.

The results showed that the in vitro binding affinity constants of the anti-human CLDN18.2 humanized antibodies were not significantly different from those of the original murine antibodies, but were lower than that of IMAB362 by one order of magnitude. See Table 4.

TABLE 4
Binding kinetics of antibodies of the present disclousre

| Antibody | ka (M−1s−1) | kd (s−1) | KD (M) |
|---|---|---|---|
| 8K13-xiIgG | 8.19E+04 | 5.73E−04 | 6.99E−09 |
| 8K13-hz24 | 7.09E+04 | 2.02E−03 | 2.85E−08 |
| 16K15-xiIgG | 1.07E+05 | 1.44E−03 | 1.35E−08 |
| 16K15-hz22 | 5.71E+04 | 5.63E−04 | 9.85E−09 |
| 52E22-xiIgG | 4.90E+05 | 5.45E−04 | 1.11E−09 |
| 52E22-hz12 | 1.46E+05 | 1.72E−04 | 1.18E−09 |
| IMAB362 | 1.48E+06 | 0.201 | 1.36E−07 |

### Example 6 Cytology Assay In Vitro of Anti-Human CLDN18.2 Humanized Antibodies

6.1 Complement Dependent Cytotoxicity (CDC)

The humanized antibodies of embodiments of the present disclosure were analyzed for their ability to induce complement-dependent cytotoxicity (CDC) on CHOK1, BxPC3 and NCI-N87 cells stably expressing human CLDN18.2 using a commercial human serum whole complement available by Quidel, Inc.

Cells were mixed with the antibody to be tested at a final concentration ranging from 250m/ml to 3.8 ng/ml. Whole human serum at a concentration of 6.25% dissolved in cell culture medium RPMI-1640 was added into the mixture which was then incubated for 3 hours at 37° C. Cytotoxicity was measured by a CCK-8 kit. Finally, absorbance at 450 nm was measured by a MD plate reader. EC50 values of the samples were calculated by plotting 4-parameter fitting curves with the absorbance values using the softmax pro? software.

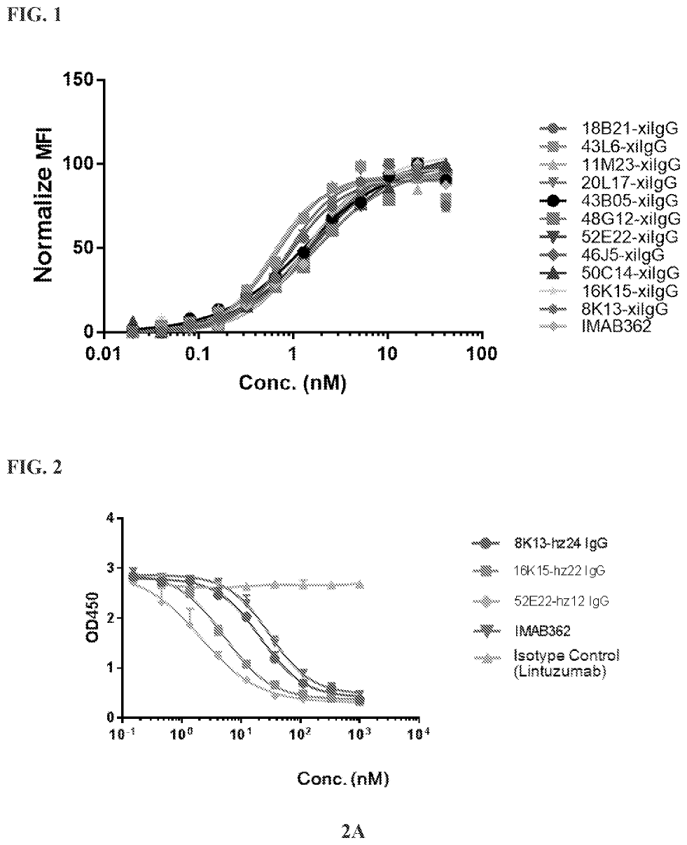

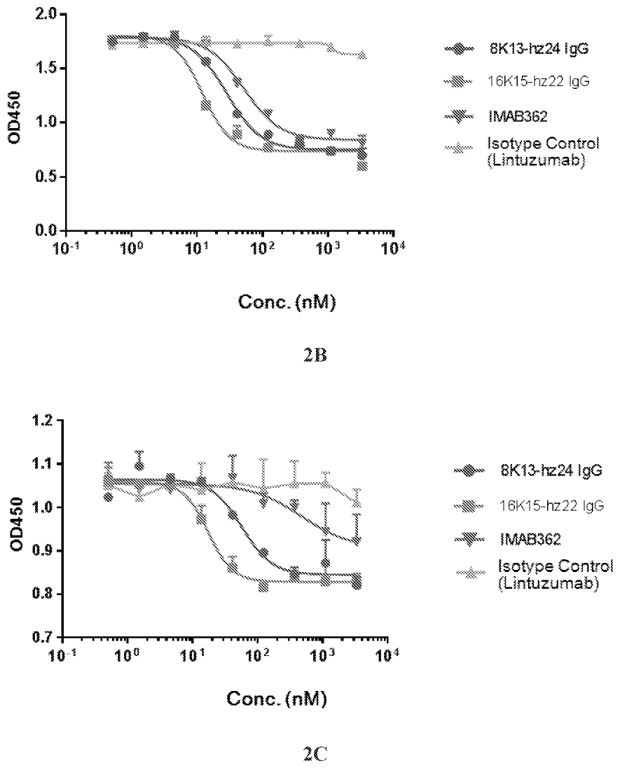

The results showed that the anti-human CLDN18.2 humanized antibodies had specific Complement Dependent Cytotoxicity (CDC) against target expressing cells and their cell killing activities were significantly superior to that of IMAB362. See FIG. 2 and Table 5.

TABLE 5
CDC of the antibodies of embodiments of the present disclosure

| Target expressing cells | a. CHOK1 | b. BxPC3 | c. NCI-N87 |
|---|---|---|---|
| Antibody | EC50 (nM) | EC50 (nM) | EC50 (nM) |
| 8K13-hz24 IgG | 21.27 | 28.53 | 57.7 |
| 16K15-hz22 IgG | 5.2 | 12.22 | 17.36 |
| 52E22-hz12 IgG | 2.08 | NA | NA |
| IMAB362 | 30.45 | 52.23 | 474.2 |

6.2 Antibody Dependent Cellular Cytotoxicity (ADCC)

Engineered Jurkat cells stably expressing a FcγRIIIa-FcεIαγ hybrid receptor and expressing firefly Luciferase under driving by the NFAT response element were used as effector cells. The biological activity of an antibody in an ADCC mechanism is quantified by Luciferase produced by activation of the NFAT pathway. 1.5E5 effector cells were mixed with the antibody to be tested at a final concentration ranging from 33 μg/ml to 85 pg/ml, and then 2.5E4 target cells were added into the mixture (effector to target E: T ratio was 6:1), which was then incubated for 16 hours at 37° C. Cytotoxicity was measured by a kit, Bio-Glo™ Luciferase Assay System from Promega; and LUM values were finally determined by a MD plate reader.

The data were processed as follows: Fold induction=(reading value of the well detected−background value)/(reading value of the negative control well−background value). EC50 values of the samples were calculated by plotting 4-parameter fitting curves with the data.

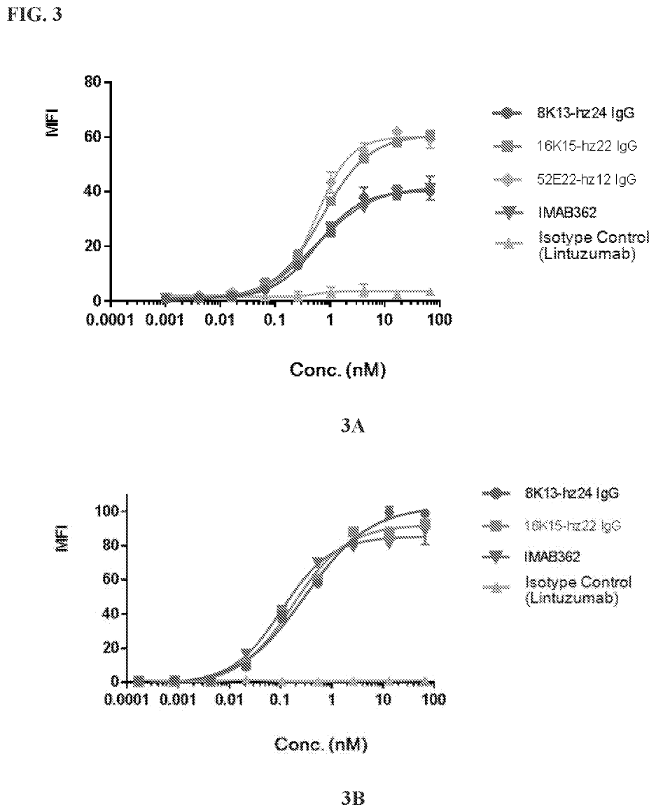

The results showed that the anti-human CLDN18.2 humanized antibodies had specific Antibody Dependent Cellular Cytotoxicity (ADCC) against target expressing cells and their cell killing activities were comparable to that of IMAB362. See FIG. 3 and Table 6.

TABLE 6
ADCC of the antibodies of embodiments of the present disclosure

| Target expressing cells | a. CHOK1 | b. BxPC3 | c. NCI-N87 |
|---|---|---|---|
| Antibody | EC50 (nM) | EC50 (nM) | EC50 (nM) |
| 8K13-hz24 IgG | 0.5894 | 0.3045 | 0.1499 |
| 16K15-hz22 IgG | 0.7122 | 0.1759 | 0.0677 |
| 52E22-hz12 IgG | 0.5672 | NA | NA |
| IMAB362 | 0.5929 | 0.1056 | 0.0909 |

### Example 7 Analysis of Binding Characteristics of the Anti-Human CLDN18.2 Humanized Antibodies to Proteins of the Same Family

The genes of human CLDN18.2 and CLDN18.1 were constructed, respectively, into eukaryotic expression vectors, and HEK293 cells were transfected with the vectors using Polyethyleneimine (PEI). Three days after the transfection, the cells were collected by centrifugation, washed with PBS once, and resuspended at a cell density of 2×10⁶/ml. 10 μl was added to each well of a 384-well plate and then humanized antibodies at different concentrations were added into the wells. After incubation for 1 hour at 4° C., fluorescently labeled secondary goat anti-human IgG antibody was added. After continued incubation for 1 hour at 4° C., the mean fluorescence readings of the 384-well plate were read by a flow cytometer. The data were analyzed to obtain the binding characteristics of the cells to the anti-human CLDN18.2 humanized antibodies. The positive control used for the experiment was the commercial anti-human CLDN18 rabbit mAb 34H14L15 (available from Abcam corporation) and the negative Isotype control was the anti-CD33 IgG antibody Lintuzumab in full-length.

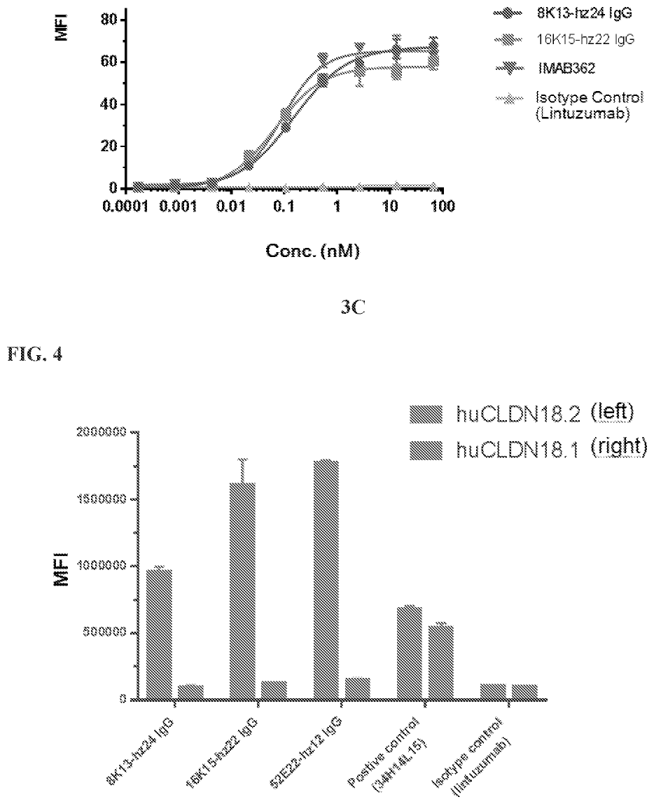

The results showed that the anti-human CLDN18.2 humanized antibodies exhibited characteristics of binding to human CLDN18.2 but not to human CLDN18.1. See FIG. 4. And the non-specific binding to human CLDN18.1 cells at low, medium and high concentrations was all lower than that of IMAB362. See FIG. 5.

### Example 8 Analysis of Human, Monkey, and Murine Species Cross Binding Characteristics of the Anti-Human CLDN18.2 Humanized Antibodies

The genes of human, murine and Rhesus CLDN18.2 were constructed, respectively, into eukaryotic expression vectors, and HEK293 cells were transfected with the vectors using Polyethyleneimine (PEI). The cells were collected 2 days later. The binding specificity of the cells to anti-human CLDN18.2 humanized antibodies was analyzed using flow cytometry. See Example 7 for the procedure of flow cytometry.

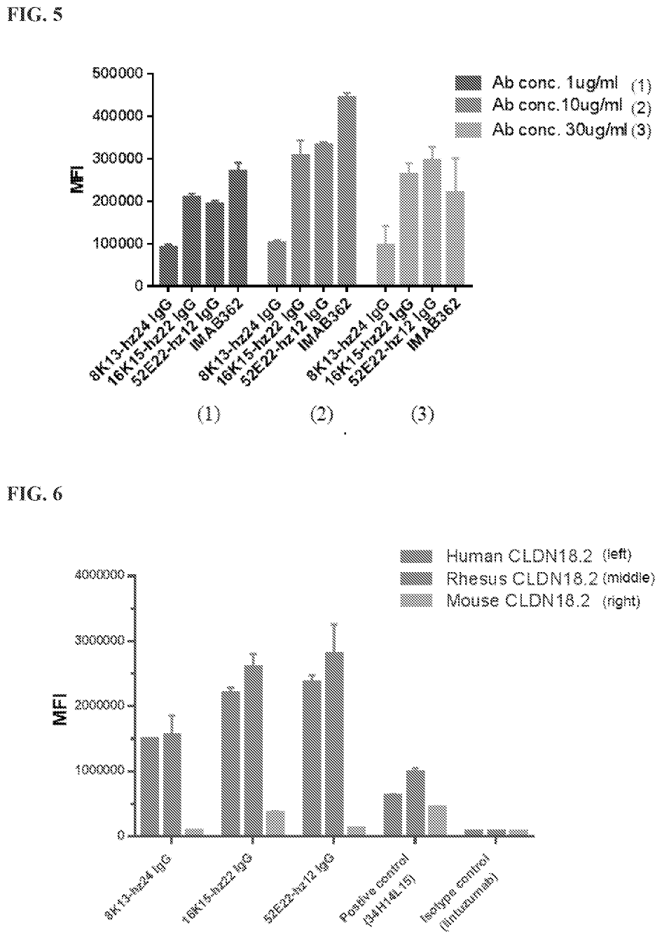

The results showed that the anti-human CLDN18.2 humanized antibody 16K15-22 IgG bound to CLDN18.2 of all three species, and 8K13-24 IgG and 52E22-12 IgG bound to Rhesus CLDN18.2 but not to mouse CLDN18.2. The results are shown in FIG. 6.

### Example 9 Preparation of CAR-T Cells Using Anti-Human CLDN18.2 Humanized Antibodies and Activation Thereof

9.1 Lentivirus Packaging

Lentivirus packaging was performed according to the grouping shown in Table 7. Lentiviral vector plasmids pLTR containing different antibody genes were first constructed, and their plasmid DNA was extracted using a plasmid extraction kit from Qiagen after the DNA was confirmed correct by sequencing. The plasmid DNA was dissolved in sterile TE, and its concentration and purity were determined by UV light absorption, ensuring A260/A280 of the extracted plasmid DNA was between 1.8 and 2.0. The DNA of two helper packaging element plasmids, pCMV-VSV-G and pCMV-dR8.2, was extracted as well. HEK293T cells for transfection were prepared to obtain freshly passaged cells which should grow to a confluency of about 60%. Three plasmids were co-transferred into HEK293T cells using calcium phosphate as a transfection reagent. 48 hours after transfection, cell supernatant containing the packaged virus was collected by centrifugation at low temperature, and cell debris was removed using a 0.45 μm filter. The virus was concentrated using an ultrafiltration centrifuge tube, and sub-packaged and stored in a refrigerator at −80° C. A small amount of the virus concentrate was taken and the virus titer was determined by FACS.

TABLE 7
Lentivirus groupings

| No. | Virus | Virus titer | Group |
|---|---|---|---|
| 135 | LV-135.N | 8.75E+06 | Negative control |
| 417 | LV-417.N | 1.65E+07 | 16k15 (16K15-hz22) scfv* |
| 418 | LV-418.N | 1.38E+07 | 52E22 (52E22-hz12) scfv* |
| 419 | LV-419.N | 5.13E+07 | 8K13 (8K13-hz24) scfv* |
| 420 | LV-420.N | 1.50E+07 | Positive control |

The negative control shown in Table 7 was another antibody not binding CLDN18.2 and the positive control was antibody hu8E5 from Carsgen Therapeutics (see WO2018006882A1). *: The single chain antibody was formed by linking humanized VH and VL via a short peptide (GST SGGGSGGGSGGGGS S).

9.2 Preparation of CAR-T Cells

A healthy donor detected negative for HBV, HCV and HIV was selected. 100 ml of blood was collected from the antecubital vein, and Ficoll density gradient centrifugation was performed to isolate the white layer containing PBMCs. CD3+ T cells were isolated using DynaBeads CD3/CD28 (LifeTechnologies, Cat. No. 40203D) at a ratio of 3:1 of DynaBeads: CD3+ T cells. 24 hours after activation, the percentage of CD25+CD69+ T cells was detected by flow cytometry. When activated, the CD3+ T cells were transduced with the lentiviruses at an MOI of 5. A 24-well plate was coated with Novonectin for 2 hours at 37° C.; the cell suspension obtained by the procedure above was formulated into a suspension for transduction with various lentiviruses (MOI=5), SYNPERONIC® F108 (Sigma, Cat. No. 07579-250G-F), and Tscm (2 U/ml). The suspension for transduction was added into the 24-well plate with the density of cells adjusted to 1.0E+06/ml. The plate was centrifuged at 500 g for 30 min, and placed in an incubator at 37° C., 5% CO₂ for static culture for 48 h. After the transduction, the cells were cultured in 5% FBS X-vivol5 medium (LONZA, Cat. No. 04-418Q), supplemented with Tscm (at a final concentration of 2 U/ml) on alternate days. The cells were counted, adjusted to 0.5E+06/ml, and harvested on Day 8-10 of culture.

The results of positive percentages of CAR-T cells in each group obtained are shown in FIG. 7.

9.3 Activation of CAR-T Cells

CHO cells expressing antigen human CLDN18.1 and antigen human CLDN18.2 were constructed and used as target cells.

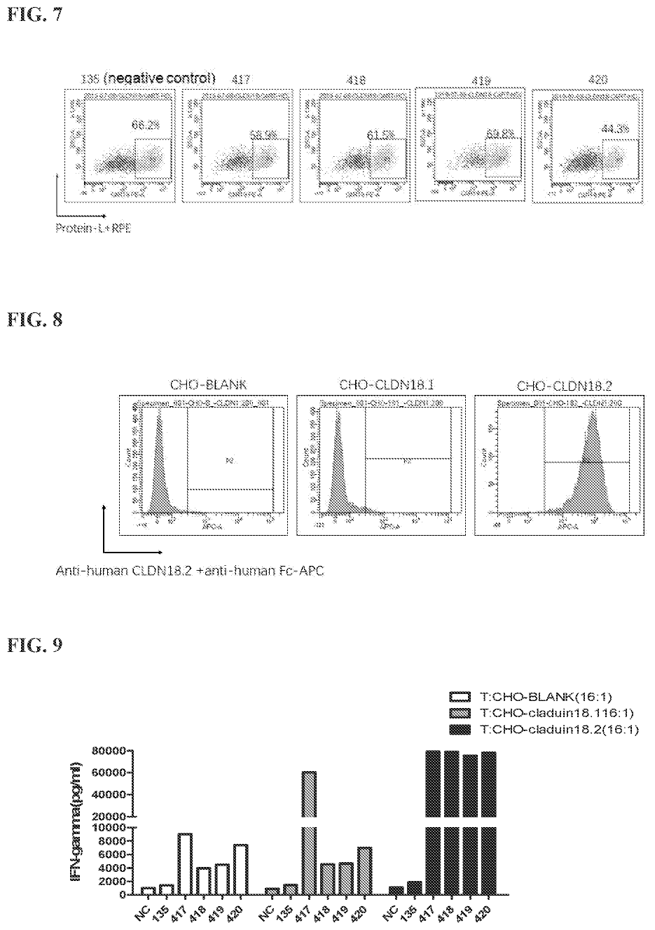

Detection results of antigen expression of the cells are shown in FIG. 8. CHO-BLANK (blank control) and CHO-CLDN18.1 had no expression of antigen CLDN18.2, and CHO-CLDN18.2 had a high expression of antigen CLDN18.2.

The effector cells with density adjusted as described above, including each group of CAR-T cells and T cells without lentivirus transduction were mixed with the target cells in 1.5 mL centrifuge tubes, respectively, at an effective to target ratio of 16:1. Total volumes were replenished to 200 μL using T cell broth X-vivol5 (free of autologous serum and Tscm); and then 200 μL of the systems was respectively transferred into a 96-well plate V-bottom for a 24-hour co-incubation.

After the incubation, the supernatant of each culture system was taken for the detection of human IFNγ. A significant increase in IFNγ release was found in the CAR-T cells in group 417 after incubation with CHO-CLDN18.1 and CHO-CLDN18.2; and an increase in IFNγ release was found in the CAR-T cells in groups 418, 419, and 420 only after incubation with CHO-CLDN18.2. The results are shown in Table 8 and FIG. 9.

TABLE 8
Human IFNγ detected in the supernatants of the culture systems

| Group | T: CHO-BLANK | T: CHO-claduin18.1 | T: CHO-claduin18.2 |
|---|---|---|---|
| nc | 1020.1 | 912 | 1095.2 |
| 135 | 1441.2 | 1458.6 | 1877 |
| 417 | 9016 | 60328.2 | 79178.6 |
| 418 | 3940.8 | 4540 | 78903.4 |
| 419 | 4486.2 | 4670.6 | 75516.8 |
| 420 | 7381.1 | 6973.2 | 78247 |

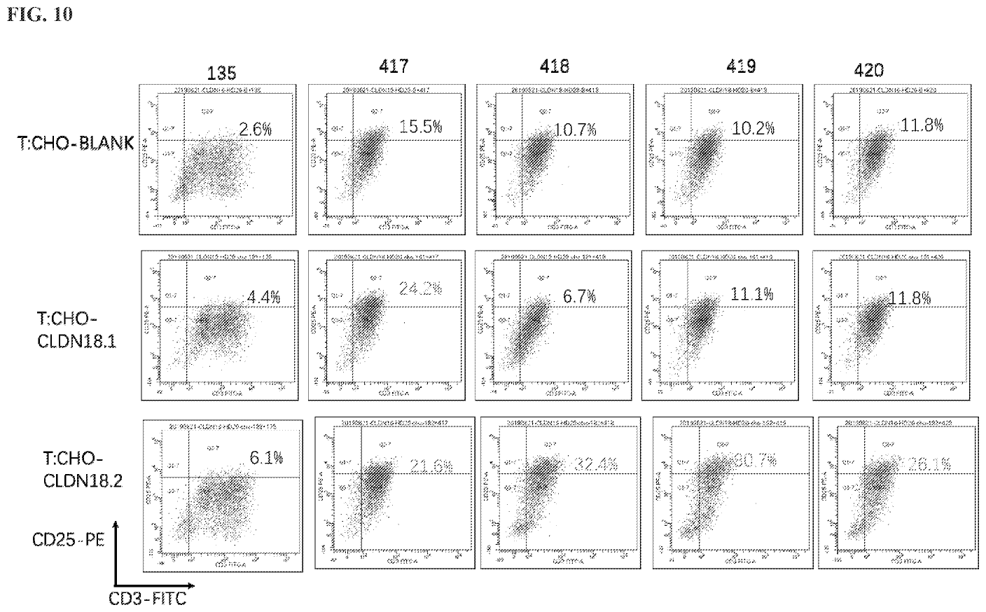

The CAR-T cells from each group were detected for T-cell activation marker proteins CD3/CD25. CD25 expression was found to be up-regulated in CAR-T cells in group 417 after incubation with CHO-CLDN18.1 and CHO-CLDN18.2, and up-regulated in CAR-T cells in groups 418, 419, and 420 CART only after incubation with CHO-CLDN18.2. See FIG. 10.

The above description of embodiments of the present disclosure is not intended to limit the present disclosure, and those skilled in the art may make various changes and modifications to the present disclosure without departing from the spirit of the present disclosure, which should fall within the scope of the appended claims.

What is claimed is:

1. A preparation method of an anti-human Claudin 18.2 antibody, the preparation method including steps as follows:
(1) immunizing an animal with cells expressing human Claudin 18.2 protein on surface as an immunogen;
(2) preparing cell clones capable of producing antibodies using the animals immunized in step (1);
(3) using cells expressing human Claudin 18.2 protein on surface as a positive screening antigen, screening antibodies having a binding activity to the positive screening antigen and cells producing the antibodies;
(4) using the cells expressing human Claudin 18.1 protein on surface as a negative screening antigen, excluding antibodies having a binding activity to the negative screening antigen and cells producing the antibody.

2. The preparation method according to claim 1, wherein the cells expressing the human Claudin 18.2 protein in step (1) are derived from the same species as the animal immunized; preferably, the cells expressing the human Claudin 18.2 protein are mouse cells, and the animal immunized is a mouse.

3. The preparation method according to claim 1, wherein the cell clones capable of producing antibodies in step (2) are prepared by a technique selected from the group consisting of hybridoma technique and single cell amplification technique.

4. The preparation method according to claim 1, wherein the positive screening antigen in step (3) is the same as the immunogen in step (1); and the negative screening antigen in step (4) differs from the immunogen in step (1) only in that the protein expressed is human Claudin 18.1 protein.

5. The preparation method according to claim 1, wherein step (3) and step (4) each is conducted by a method selected from the group consisting of Enzyme-Linked Immunosorbent Assay (ELISA) and Fluorescence Resonance Energy Transfer (FRET).

6. An anti-human Claudin 18.2 antibody obtained according to the preparation method of any one of claims 1 to 6.

7. The anti-human Claudin 18.2 antibody according to claim 6, which specifically binds the N-terminus of human Claudin 18.2, preferably extracellular region at the N-terminus of human Claudin 18.2 comprising the first Extracellular Loop (ECL1).

8. The anti-human Claudin 18.2 antibody according to claim 6, which has a specific binding to human CLDN18.2 at nM scale; and which has no significant difference in binding to human CLDN18.1 as compared to an isotype negative antibody (or an unrelated antibody).

9. An antibody or fragment thereof comprising a heavy chain variable region (VH) and a light chain variable region (VL), wherein the heavy chain variable region (VH) and the light chain variable region (VL) comprise a combination of CDRs (VH-CDR1, VH-CDR2, VH-CDR3; and VL-CDR1, VL-CDR2, VL-CDR3) selected from the group consisting of:
(1) VH-CDR1 as shown in SEQ ID NO. 31, VH-CDR2 as shown in SEQ ID NO. 32, and VH-CDR3 as shown in SEQ ID NO. 33; and VL-CDR1 as shown in SEQ ID NO. 34, VL-CDR2 as shown in SEQ ID NO. 35, and VL-CDR3 as shown in SEQ ID NO. 36;
(2) VH-CDR1 as shown in SEQ ID NO. 37, VH-CDR2 as shown in SEQ ID NO. 38, and VH-CDR3 as shown in SEQ ID NO. 39, and VL-CDR1 as shown in SEQ ID NO. 40, VL-CDR2 as shown in SEQ ID NO. 41, and VL-CDR3 as shown in SEQ ID NO. 42,
(3) VH-CDR1 as shown in SEQ ID NO. 43, VH-CDR2 as shown in SEQ ID NO. 44, and VH-CDR3 as shown in SEQ ID NO. 45; and VL-CDR1 as shown in SEQ ID NO. 46, VL-CDR2 as shown in SEQ ID NO. 41, and VL-CDR3 as shown in SEQ ID NO. 47;
(4) VH-CDR1 as shown in SEQ ID NO. 48, VH-CDR2 as shown in SEQ ID NO. 49, and VH-CDR3 as shown in SEQ ID NO. 50; and VL-CDR1 as shown in SEQ ID NO. 40, VL-CDR2 as shown in SEQ ID NO. 41, and VL-CDR3 as shown in SEQ ID NO. 51;
(5) VH-CDR1 as shown in SEQ ID NO. 52, VH-CDR2 as shown in SEQ ID NO. 53, and VH-CDR3 as shown in SEQ ID NO. 54; and VL-CDR1 as shown in SEQ ID NO. 55, VL-CDR2 as shown in SEQ ID NO. 41, and VL-CDR3 as shown in SEQ ID NO. 56;
(6) VH-CDR1 as shown in SEQ ID NO. 57, VH-CDR2 as shown in SEQ ID NO. 58, and VH-CDR3 as shown in SEQ ID NO. 33; and VL-CDR1 as shown in SEQ ID NO. 34, VL-CDR2 as shown in SEQ ID NO. 59, and VL-CDR3 as shown in SEQ ID NO. 60;
(7) VH-CDR1 as shown in SEQ ID NO. 61, VH-CDR2 as shown in SEQ ID NO. 62, and VH-CDR3 as shown in SEQ ID NO. 63; and VL-CDR1 as shown in SEQ ID NO. 46, VL-CDR2 as shown in SEQ ID NO. 41, and VL-CDR3 as shown in SEQ ID NO. 64;
(8) VH-CDR1 as shown in SEQ ID NO. 65, VH-CDR2 as shown in SEQ ID NO. 66, and VH-CDR3 as shown in SEQ ID NO. 67; and VL-CDR1 as shown in SEQ ID NO. 68, VL-CDR2 as shown in SEQ ID NO. 41, and VL-CDR3 as shown in SEQ ID NO. 69;
(9) VH-CDR1 as shown in SEQ ID NO. 65, VH-CDR2 as shown in SEQ ID NO. 70, and VH-CDR3 as shown in SEQ ID NO. 71; and VL-CDR1 as shown in SEQ ID NO. 72, VL-CDR2 as shown in SEQ ID NO. 41, and VL-CDR3 as shown in SEQ ID NO. 73;
(10) VH-CDR1 as shown in SEQ ID NO. 74, VH-CDR2 as shown in SEQ ID NO. 75, and VH-CDR3 as shown in SEQ ID NO. 76; and VL-CDR1 as shown in SEQ ID NO. 77, VL-CDR2 as shown in SEQ ID NO. 41, and VL-CDR3 as shown in SEQ ID NO. 78;
(11) VH-CDR1 as shown in SEQ ID NO. 79, VH-CDR2 as shown in SEQ ID NO. 80, and VH-CDR3 as shown in SEQ ID NO. 81; and VL-CDR1 as shown in SEQ ID NO. 82, VL-CDR2 as shown in SEQ ID NO. 41, and VL-CDR3 as shown in SEQ ID NO. 83;
(12) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 42;
(13) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 86;
(14) VH-CDR1 as shown in SEQ ID NO. 37, VH-CDR2 as shown in SEQ ID NO. 38, and VH-CDR3 as shown in SEQ ID NO. 39; and VL-CDR1 as shown in SEQ ID NO. 85, VL-CDR2 as shown in SEQ ID NO. 41, and VL-CDR3 as shown in SEQ ID NO. 87;
(15) VH-CDR1 as shown in SEQ ID NO. 74, VH-CDR2 as shown in SEQ ID NO. 75, and VH-CDR3 as shown in SEQ ID NO. 76; and VL-CDR1 as shown in SEQ ID NO. 89, VL-CDR2 as shown in SEQ ID NO. 41, and VL-CDR3 as shown in SEQ ID NO. 90, and
(16) VH-CDR1 as shown in SEQ ID NO. 79, VH-CDR2 as shown in SEQ ID NO. 91, and VH-CDR3 as shown in SEQ ID NO. 81; and VL-CDR1 as shown in SEQ ID NO. 92, VL-CDR2 as shown in SEQ ID NO. 41, and VL-CDR3 as shown in SEQ ID NO. 93.

10. The antibody or fragment thereof according to any one of claims 6 to 9, wherein the heavy chain variable region comprises an amino acid sequence as shown in any one of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 27, and SEQ ID NO. 29 or an amino acid sequence having at least 75% identity to the amino acid sequence as shown; and/or the light chain variable region comprises an amino acid sequence as shown in any one of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 25, SEQ ID NO. 26, SEQ ID NO. 28, and SEQ ID NO. 30 is or an amino acid sequence having at least 75% identity to the amino acid sequence as shown

11. The antibody or fragment thereof according to any one of claims 6 to 10, wherein the antibody or fragment thereof comprises a heavy chain variable region and a light chain variable region selected from the group consisting of:
(1) an amino acid sequence as shown in SEQ ID NO. 1 or an amino acid sequence having at least 75% identity to the amino acid sequence as shown in SEQ ID NO. 1, and an amino acid sequence as shown in SEQ ID NO. 2 or an amino acid sequence having at least 75% identity to the amino acid sequence as shown in SEQ ID NO. 2;
(2) an amino acid sequence as shown in SEQ ID NO. 3 or an amino acid sequence having at least 75% identity to the amino acid sequence as shown in SEQ ID NO. 3; and an amino acid sequence as shown in SEQ ID NO. 4 or an amino acid sequence having at least 75% identity to the amino acid sequence as shown in SEQ ID NO. 4;
(3) an amino acid sequence as shown in SEQ ID NO. 5 or an amino acid sequence having at least 75% identity to the amino acid sequence as shown in SEQ ID NO. 5; and an amino acid sequence as shown in SEQ ID NO. 6 or an amino acid sequence having at least 75% identity to the amino acid sequence as shown in SEQ ID NO. 6;
(4) an amino acid sequence as shown in SEQ ID NO. 7 or an amino acid sequence having at least 75% identity to the amino acid sequence as shown in SEQ ID NO. 7; and an amino acid sequence as shown in SEQ ID NO. 8 or an amino acid sequence having at least 75% identity to the amino acid sequence as shown in SEQ ID NO. 8;
(5) an amino acid sequence as shown in SEQ ID NO. 9 or an amino acid sequence having at least 75% identity to the amino acid sequence as shown in SEQ ID NO. 9; and an amino acid sequence as shown in SEQ ID NO. 10 or an amino acid sequence having at least 75% identity to the amino acid sequence as shown in SEQ ID NO. 10;
(6) an amino acid sequence as shown in SEQ ID NO. 11 or an amino acid sequence having at least 75% identity to the amino acid sequence as shown in SEQ ID NO. 11; and an amino acid sequence as shown in SEQ ID NO. 12 or an amino acid sequence having at least 75% identity to the amino acid sequence as shown in SEQ ID NO. 12;
(7) an amino acid sequence as shown in SEQ ID NO. 13 or an amino acid sequence having at least 75% identity to the amino acid sequence as shown in SEQ ID NO. 13; and an amino acid sequence as shown in SEQ ID NO. 14 or an amino acid sequence having at least 75% identity to the amino acid sequence as shown in SEQ ID NO. 14;
(8) an amino acid sequence as shown in SEQ ID NO. 15 or an amino acid sequence having at least 75% identity to the amino acid sequence as shown in SEQ ID NO. 15; and an amino acid sequence as shown in SEQ ID NO. 16 or an amino acid sequence having at least 75% identity to the amino acid sequence as shown in SEQ ID NO. 16;
(9) an amino acid sequence as shown in SEQ ID NO. 17 or an amino acid sequence having at least 75% identity to the amino acid sequence as shown in SEQ ID NO. 17; and an amino acid sequence as shown in SEQ ID NO. 18 or an amino acid sequence having at least 75% identity to the amino acid sequence as shown in SEQ ID NO. 18;
(10) an amino acid sequence as shown in SEQ ID NO. 19 or an amino acid sequence having at least 75% identity to the amino acid sequence as shown in SEQ ID NO. 19; and an amino acid sequence as shown in SEQ ID NO. 20 or an amino acid sequence having at least 75% identity to the amino acid sequence as shown in SEQ ID NO. 20;
(11) an amino acid sequence as shown in SEQ ID NO. 21 or an amino acid sequence having at least 75% identity to the amino acid sequence as shown in SEQ ID NO. 21; and an amino acid sequence as shown in SEQ ID NO. 22 or an amino acid sequence having at least 75% identity to the amino acid sequence as shown in SEQ ID NO. 22;
(12) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 24 or an amino acid sequence having at least 75% identity to the amino acid sequence as shown in SEQ ID NO. 24;
(13) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 25 or an amino acid sequence having at least 75% identity to the amino acid sequence as shown in SEQ ID NO. 25;
(14) an amino acid sequence as shown in SEQ ID NO. 23 or an amino acid sequence having at least 75% identity to the amino acid sequence as shown in SEQ ID NO. 23; and an amino acid sequence as shown in SEQ ID NO. 26 or an amino acid sequence having at least 75% identity to the amino acid sequence as shown in SEQ ID NO. 26;
(15) an amino acid sequence as shown in SEQ ID NO. 27 or an amino acid sequence having at least 75% identity to an amino acid sequence as set forth in SEQ ID NO. 27; and an amino acid sequence as shown in SEQ ID NO. 28 or an amino acid sequence having at least 75% identity to the amino acid sequence as shown in SEQ ID NO. 28;
(16) an amino acid sequence as shown in SEQ ID NO. 29 or an amino acid sequence having at least 75% of identity to the amino acid sequence as shown in SEQ ID NO. 29; and an amino acid sequence as shown in SEQ ID NO. 30 or an amino acid sequence having at least 75% of identity to the amino acid sequence as shown in SEQ ID NO. 30.

12. The antibody or fragment thereof according to any one of claims 6 to 11, wherein the antibody or fragment thereof is in any form, e.g., a monoclonal antibody, a single chain antibody, a diabody, a single domain antibody, a nanobody, a fully or partially humanized antibody, or a chimeric antibody and the like; alternatively, the antibody or fragment thereof is a half-antibody or an antigen-binding fragment of the half-antibody, e.g., scFv, BsFv, dsFv, (dsFv)₂, Fab, Fab′, F(ab′)₂, or Fv;
preferably, the antibody or fragment thereof further comprises a human or murine constant region, preferably a human or murine light chain constant region (CL) and/or heavy chain constant region (CH);
more preferably, the antibody or fragment thereof comprises a heavy chain constant region selected from the group consisting of IgG, IgA, IgM, IgD and IgE and/or a kappa or lambda type light chain constant region.

13. The antibody or fragment thereof according to any one of claims 6 to 12, wherein the antibody is a monoclonal antibody, preferably a murine, chimeric, or humanized monoclonal antibody;
preferably, the heavy chain constant region of the monoclonal antibody is of IgG1 or IgG4 subtype and the light chain constant region of the monoclonal antibody is of kappa type;
preferably, the heavy chain constant region of the monoclonal antibody comprises an amino acid sequence as shown in SEQ ID NO: 124 or an amino acid sequence having at least 75% identity to the amino acid sequence as shown;
preferably, the light chain constant region of the monoclonal antibody comprises an amino acid sequence as shown in SEQ ID NO: 125 or an amino acid sequence having at least 75% identity to the amino acid sequence as shown.

14. A nucleic acid molecule comprising a nucleotide sequence encoding the antibody or fragment thereof according to any one of claims 6 to 13, or encoding a heavy chain CDR, a light chain CDR, a heavy chain variable region, a light chain variable region, a heavy chain or a light chain comprised in the antibody or fragment thereof.

15. A vector comprising the nucleic acid molecule according to claim 14.

16. A host cell comprising the nucleic acid molecule according to claim 14 and/or the vector according to claim 15, or transformed or transfected with the nucleic acid molecule according to claim 14 and/or the vector according to claim 15.

17. A conjugate or fusion protein comprising the antibody or fragment thereof according to any one of claims 6 to 13.

18. A pharmaceutical composition comprising the antibody or fragment thereof according to any one of claims 6 to 13, the nucleic acid molecule according to claim 14, the vector according to claim 15, the host cell according to claim 16, and/or the conjugate or fusion protein according to claim 17, and optionally a pharmaceutically acceptable excipient.

19. A kit comprising the antibody or fragment thereof according to any one of claims 6 to 13, the nucleic acid molecule according to claim 14, the vector according to claim 15, the host cell according to claim 16, the conjugate or fusion protein according to claim 17, and/or the pharmaceutical composition according to claim 18.

20. Use of the antibody or fragment thereof according to any one of claims 6 to 13, the nucleic acid molecule according to claim 14, the vector according to claim 15, the host cell according to is claim 16, the pharmaceutical composition according to claim 17, and/or the conjugate or fusion protein according to claim 18 in the manufacture of a medicament for the prevention and/or treatment of cancer.

21. Use of the antibody or fragment thereof according to any one of claims 6 to 13, the nucleic acid molecule according to claim 14, the vector according to claim 15, the host cell according to claim 16, the pharmaceutical composition according to claim 17, and/or the conjugate or fusion protein according to claim 18 in the manufacture of an agent for the diagnosis of cancer.

22. Use of the antibody or fragment thereof according to any one of claims 6 to 13, the nucleic acid molecule according to claim 14, the vector according to claim 15, the host cell according to claim 16, the pharmaceutical composition according to claim 17, and/or the conjugate or fusion protein according to claim 18 in the manufacture of CAR-T cells.

23. A method for preventing and/or treating cancer, the method including administering to a subject in need thereof the antibody or fragment thereof according to any one of claims 6 to 13, the nucleic acid molecule according to claim 14, the vector according to claim 15, the host cell according to claim 16, the pharmaceutical composition according to claim 17, and/or the conjugate or fusion protein according to claim 18, and optionally other drugs or means.

24. A method for diagnosing cancer, the method including contacting the antibody or fragment thereof according to any one of claims 6 to 13, the nucleic acid molecule according to claim 14, the vector according to claim 15, the host cell according to claim 16, the pharmaceutical composition according to claim 17, and/or the conjugate or fusion protein according to claim 18 with a sample from a subject.
